SEQUENCE LISTING (10 sequences)

SEQ ID NO: 1 - protein, 449 aa, Artificial Sequence
  Synthetic Construct
QITLKESGPTLVKPTQTLTLTCTFSGFSLNTYAMGVSWIRQPPGKALEWLASIWWNGNKYNNPSLKSRLTVTKDTSKNQVVLTMTNMDPVDTATYYCAHSRIIRFTDYVMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 2 - protein, 214 aa, Artificial Sequence
  Synthetic Construct
DIQMTQSPSSLSTSVGDRVTITCRASEDIYNDLAWYQQKPGKAPKLLIYNANSLHTGVASRFSGSGSGTDFTFTISSLQPEDVATYFCQQYYDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 123 aa, Artificial Sequence
  Synthetic Construct
QITLKESGPTLVKPTQTLTLTCTFSGFSLNTYAMGVSWIRQPPGKALEWLASIWWNGNKYNNPSLKSRLTVTKDTSKNQVVLTMTNMDPVDTATYYCAHSRIIRFTDYVMDAWGQGTLVTVSS

SEQ ID NO: 4 - protein, 107 aa, Artificial Sequence
  Synthetic Construct
DIQMTQSPSSLSTSVGDRVTITCRASEDIYNDLAWYQQKPGKAPKLLIYNANSLHTGVASRFSGSGSGTDFTFTISSLQPEDVATYFCQQYYDYPLTFGQGTKLEIK

SEQ ID NO: 5 - protein, 7 aa, Artificial Sequence
  Synthetic Construct
TYAMGVS

SEQ ID NO: 6 - protein, 16 aa, Artificial Sequence
  Synthetic Construct
SIWWNGNKYNNPSLKS

SEQ ID NO: 7 - protein, 13 aa, Artificial Sequence
  Synthetic Construct
SRIIRFTDYVMDA

SEQ ID NO: 8 - protein, 11 aa, Artificial Sequence
  Synthetic Construct
RASEDIYNDLA

SEQ ID NO: 9 - protein, 7 aa, Artificial Sequence
  Synthetic Construct
NANSLHT

SEQ ID NO: 10 - protein, 9 aa, Artificial Sequence
  Synthetic Construct
QQYYDYPLT

(19) United States
(12) Patent Application Publication
(10) Pub. No.: US 2022/0289847 A1
(43) Pub. Date: Sep. 15, 2022

(54) METHODS FOR THE TREATMENT OF CANCER

(71) Applicant: Jounce Therapeutics, Inc., Cambridge, MA (US)

(72) Inventors: Lara Lewis MCGRATH, Arlington, MA (US); Amy Jiayue MUELLER, Cambridge, MA (US); Karen Suzanne BROWN, Lawrence Township, NJ (US); Benjamin Richards UMIKER, Watertown, MA (US); Yasmin Lucy RAMSAY, Watertown, MA (US)

(21) Appl. No.: 17/690,634

(22) Filed: Mar. 9, 2022

Related U.S. Application Data
(60) Provisional application No. 63/225,680, filed on Jul. 26, 2021.
(60) Provisional application No. 63/213,426, filed on Jun. 22, 2021.
(60) Provisional application No. 63/158,768, filed on Mar. 9, 2021.

(51) Int. Cl. C07K 16/28; G01N 33/574; C12Q 1/6886; A61K 39/395; A61P 35/00

(52) U.S. Cl. CPC C07K 16/2818; G01N 33/57488; C12Q 1/6886; C07K 16/2803; A61K 39/39533; A61P 35/00; G01N 2333/70503; G01N 2333/57; C12Q 2600/106; C07K 2317/565; A61K 2039/507

(57) ABSTRACT

The invention provides methods of treating cancer and methods for selecting approaches for treatment of cancer.

### SEQUENCE LISTING

The instant application contains a Sequence Listing which has been submitted electronically in ASCII format and is hereby incorporated by reference in its entirety. Said ASCII copy, created on Mar. 9, 2022, is named 51266-017004_Sequence_Listing_3_8_22_ST25 and is 9,611 bytes in size.

### FIELD OF THE INVENTION

The invention relates to methods of treating cancer and methods for selecting approaches for treatment of cancer.

### BACKGROUND

Myeloid cells, such as dendritic cells and macrophages, can instruct the adaptive immune system to mount a response against tumor cells and pathogens by presenting peptide antigens to T cells while expressing immunogenic cytokines and costimulatory signals, thereby promoting cytotoxic T cell activation and proliferation. Conversely, in a steady state condition, myeloid cells maintain tolerance to endogenous proteins by presenting self-antigens to T cells in the context of non-immunogenic signals, such as regulatory cytokines, which can promote regulatory T cells and suppress immunogenicity.

Cancer cells can evade the immune system by engaging signaling pathways associated with immunosuppressive or immunoregulatory antigen presentation. Such evasion events represent a major obstacle to therapeutic strategies that rely on promoting anti-tumor immunity. Therefore, there is a need for therapeutic compositions and methods that prevent tumor-induced immunosuppression and promote immunogenic presentation of tumor antigens by myeloid cells. Furthermore, there is a need for methods to select approaches for treating cancer.

### SUMMARY

The invention features methods, kits, and compositions for treating cancer and for selecting approaches for treating cancer.

In one aspect, the invention provides a method of treating cancer in a subject, the method comprising determining the ratio of LILRB2 to IFNg in a sample from the subject and, if elevated LILRB2 relative to IFNg is detected, administering a LILRB2 antibody and a PD1 antagonist to the subject.

In another aspect, the invention provides a method of treating cancer in a subject, the method comprising determining the ratio of LILRB2 to IFNg in a sample from the subject and, if a balanced level of LILRB2 and IFNg, or elevated IFNg relative to LILRB2, is detected, administering a PD1 antagonist to the subject in the absence of a LILRB2 antibody.

In another aspect, the invention provides a method for treating cancer in a subject, the method comprising administering a LILRB2 antibody and a PD1 antagonist to the subject, wherein elevated LILRB2 relative to IFNg has been detected in a sample from the subject.

In another aspect, the invention provides a method of treating cancer in a subject, the method comprising administering a PD1 antagonist to the subject, in the absence of a LILRB2 antibody, wherein a balanced level of LILRB2 and IFNg, or elevated IFNg relative to LILRB2, has been detected in a sample from the subject.

In another aspect, the invention provides a method for identifying a subject whose cancer is likely to have an improved response to combination therapy with a LILRB2 antibody and a PD1 antagonist, the method comprising determining the ratio of LILRB2 to IFNg in a sample from the subject, wherein detection of elevated LILRB2 relative to IFNg indicates that a subject is likely to have an improved response to the combination therapy.

In another aspect, the invention provides a method for identifying a subject whose cancer is likely to respond to a PD1 antagonist, without improvement by combination treatment with a LILRB2 antibody, the method comprising determining the ratio of LILRB2 to IFNg in a sample from the subject, wherein detection of a balanced level of LILRB2 and IFNg, or elevated IFNg relative to LILRB2, indicates that a subject is likely to respond to a PD1 antagonist, without improvement by combination treatment with a LILRB2 antibody.

In another aspect, the invention provides a method for selecting a cancer therapy for a subject, the method comprising determining the ratio of LILRB2 to IFNg in a sample from the subject, wherein detection of elevated LILRB2 relative to IFNg indicates selection of a LILRB2 antibody and a PD1 antagonist for treatment of the subject.

In another aspect, the invention provides a method for selecting a cancer therapy for a subject, the method comprising determining the ratio of LILRB2 to IFNg in a sample from the subject, wherein detection of a balanced level of LILRB2 and IFNg, or elevated IFNg relative to LILRB2, indicates selection of a PD1 antagonist for treatment of the subject, in the absence of a LILRB2 antibody.

In another aspect, the invention provides a method of improving the response of a subject to PD1 antagonist cancer therapy, the method comprising administering a LILRB2 antibody to the subject, wherein elevated LILRB2 relative to IFNg has been detected in a sample from the subject.

In some embodiments, the methods further comprise administering a LILRB2 antibody and a PD1 antagonist to the subject.

In some embodiments, the methods further comprise administering a PD1 antagonist to the subject, in the absence of a LILRB2 antibody.

In some embodiments, the methods further comprise administering a PD1 antagonist to the subject.

In some embodiments, the therapy comprises administration of the LILRB2 antibody and the PD1 antagonist at about the same time as one another.

In some embodiments, the combination therapy comprises administration of the LILRB2 antibody before the PD1 antagonist.

In some embodiments, detection of LILRB2 and/or IFNg levels is carried out by detection of LILRB2 and/or IFNg RNA levels.

In some embodiments, detection of LILRB2 and/or IFNg levels is carried out by detection of LILRB2 and/or IFNg protein levels.

In some embodiments, detection of LILRB2 and/or IFNg levels is carried out by detection of a LILRB2 signature, which optionally is a tumor-associated macrophage (TAM) gene signature, and/or IFNg gene signature.

In some embodiments, the sample comprises a tumor biopsy. In some embodiments, the sample comprises a blood sample, such as a peripheral blood sample or a sample comprising peripheral blood mononuclear cells.

In some embodiments, the LILRB2 antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

In some embodiments, the antibody comprises a V_{H} region comprising an amino acid sequence that is at least 90% identical to the amino acid sequence of SEQ ID NO: 3 and a V_{L} region comprising an amino acid sequence that is at least 90% identical to the amino acid sequence of SEQ ID NO: 4, wherein the V_{H} region comprises three CDRs comprising the amino acid sequences of SEQ ID NOs: 5-7, and the V_{L} region comprises three CDRs comprising the amino acid sequences of SEQ ID NOs: 8-10.

In some embodiments, the antibody comprises a V_{H} region comprising the amino acid sequence of SEQ ID NO: 3 and a variable light chain V_{L} region comprising the amino acid sequence of SEQ ID NO: 4.

In some embodiments, the antibody comprises a heavy chain comprising the amino acid sequence of SEQ ID NO: 1 and a light chain comprising the amino acid sequence of SEQ ID NO: 2.

In some embodiments, the LILRB2 antibody is selected from the group consisting of: JTX-8064, MK-4830, NGM707, IO-108, and iosH2.

In some embodiments, the PD1 antagonist is directed against PD1.

In some embodiments, the PD1 antagonist is directed against PD-L1.

In some embodiments, the PD1 antagonist comprises an antibody.

In some embodiments, the PD1 antagonist antibody is selected from the group consisting of: JTX-4014, nivolumab, pidilizumab, lambrolizumab, pembrolizumab, cemiplimab, avelumab, atezolizumab tislelizumab, durvalumab, spartalizumab, genolimzumab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1.

In some embodiments, the PD1 antagonist antibody is JTX-4014.

In some embodiments, the cancer of the subject is selected from the group consisting of gastric cancer, melanoma (e.g., skin cutaneous melanoma), urothelial cancer, lymphoid neoplasm, diffuse large B-cell lymphoma (DLBCL), testicular germ cell tumors (TGCT), mesothelioma, kidney cancer (e.g., kidney renal clear cell carcinoma, kidney renal papillary cell carcinoma, or renal cell carcinoma (RCC)), sarcoma, lung cancer (e.g., lung adenocarcinoma, lung squamous cell carcinoma, and non-small cell lung cancer (NSCLC)) stomach adenocarcinoma, pancreatic adenocarcinoma, head and neck squamous cell carcinoma, ovarian serious cystadenocarcinoma, liver hepatocellular carcinoma, skin cutaneous melanoma, colon adenocarcinoma, breast cancer (e.g., breast invasive carcinoma or triple negative breast cancer), rectum adenocarcinoma, glioblastoma multiforme, uterine corpus endometrial carcinoma, thymoma, bladder cancer, endometrial cancer, Hodgkin's lymphoma, ovarian cancer, anal cancer, biliary cancer, colorectal cancer, and esophageal cancer.

In some embodiments, the cancer of the subject is gastric cancer, melanoma, or urothelial cancer.

In some embodiments, the method further comprises administration of an additional therapeutic agent to the subject. In some embodiments, the additional therapeutic agent is an anti-cancer agent, e.g., as described herein.

In some embodiments, the subject is a human patient.

In another aspect, the invention provides a kit for use in determining whether to administer a combination of a LILRB2 antibody and a PD1 antagonist to a subject having cancer according to a method described herein, the kit comprising primers, probes, and/or antibodies for detecting the level of LILRB2 RNA or protein, IFNg RNA or protein, and/or the components of a gene signature for LILRB2 and/or IFNg in a sample from the subject. In some embodiments, the sample is a tumor biopsy or a peripheral blood sample (e.g., a sample comprising peripheral blood mononuclear cells).

In another aspect, the invention provides a composition comprising a unit dose of an antibody that specifically binds to human LILRB2, wherein the unit dose is 300 to 1200 mg, e.g., 400 to 900 mg or 450 to 900 mg, and the antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

In some embodiments, the antibody comprises a V_{H} region comprising an amino acid sequence that is at least 90% identical to the amino acid sequence of SEQ ID NO: 3 and a V_{L} region comprising an amino acid sequence that is at least 90% identical to the amino acid sequence of SEQ ID NO: 4, wherein the V_{H} region comprises three CDRs comprising the amino acid sequences of SEQ ID NOs: 5-7, and the V_{L} region comprises three CDRs comprising the amino acid sequences of SEQ ID NOs: 8-10.

In some embodiments, the antibody comprises a V_{H} region comprising the amino acid sequence of SEQ ID NO: 3 and a variable light chain V_{L} region comprising the amino acid sequence of SEQ ID NO: 4.

In some embodiments, the antibody comprises a heavy chain comprising the amino acid sequence of SEQ ID NO: 1 and a light chain comprising the amino acid sequence of SEQ ID NO: 2.

In some embodiments, the dose is within a range selected from the group consisting of: 300-450 mg, 350-500 mg, 400-550 mg, 450-600 mg, 500-650 mg, 550-700 mg, 600-750 mg, 650-800 mg, 700-850 mg, 750-900 mg, 800-950 mg, 850-1000 mg, 900-1050 mg, and 950-1200 mg.

In some embodiments, the dose is within a range selected from the group consisting of: 300-400 mg, 350-450 mg, 400-500 mg, 450-550 mg, 500-600 mg, 550-650 mg, 600-700 mg, 650-750 mg, 700-800 mg, 750-850 mg, 800-900 mg, 850-950 mg, 900-1000 mg, 950-1050 mg, 1000-1100 mg, 1050-1150 mg, and 1100-1200 mg.

In some embodiments, the dose is selected from the group consisting of: 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, and 1200 mg.

In some embodiments, the dose is within the range of 600-800 mg.

In some embodiments, the dose is 700 mg.

In another aspect, the invention provides a method of treating cancer in a subject, the method comprising administering an antibody that specifically binds to LILRB2 to the subject at a dose of 300 to 1200 mg, e.g., 400 to 900 mg or 450 to 900 mg, wherein the antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

In some embodiments, the dose is within a range selected from the group consisting of: 300-450 mg, 350-500 mg, 400-550 mg, 450-600 mg, 500-650 mg, 550-700 mg, 600-750 mg, 650-800 mg, 700-850 mg, 750-900 mg, 800-950 mg, 850-1000 mg, 900-1050 mg, and 950-1200 mg.

In some embodiments, the dose is within a range selected from the group consisting of: 300-400 mg, 350-450 mg, 400-500 mg, 450-550 mg, 500-600 mg, 550-650 mg, 600-700 mg, 650-750 mg, 700-800 mg, 750-850 mg, 800-900 mg, 850-950 mg, 900-1000 mg, 950-1050 mg, 1000-1100 mg, 1050-1150 mg, and 1100-1200 mg.

In some embodiments, the dose is selected from the group consisting of: 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, and 1200 mg.

In some embodiments, the dose is within the range of 600-800 mg

In some embodiments, the dose is 700 mg.

In some embodiments, the antibody comprises a V_{H} region comprising an amino acid sequence that is at least 90% identical to the amino acid sequence of SEQ ID NO: 3 and a V_{L} region comprising an amino acid sequence that is at least 90% identical to the amino acid sequence of SEQ ID NO: 4, wherein the V_{H} region comprises three CDRs comprising the amino acid sequences of SEQ ID NOs: 5-7, and the V_{L} region comprises three CDRs comprising the amino acid sequences of SEQ ID NOs: 8-10.

In some embodiments, the antibody comprises a V_{H} region comprising the amino acid sequence of SEQ ID NO: 3 and a variable light chain V_{L} region comprising the amino acid sequence of SEQ ID NO: 4.

In some embodiments, the antibody comprises a heavy chain comprising the amino acid sequence of SEQ ID NO: 1 and a light chain comprising the amino acid sequence of SEQ ID NO: 2. In some embodiments, the subject is a human patient.

In another aspect, the invention provides a method of treating cancer in a subject, the method comprising administering an antibody that specifically binds to LILRB2 to the subject at a dose of 5-15 mg/kg, wherein the antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

In some embodiments, the dose is within a range selected from the group consisting of: 5-10 mg/kg, 7.5-12.5 mg/kg, and 10-15 mg/kg.

In some embodiments, a dose of the antibody is administered once every three weeks.

In some embodiments, the method further comprises administering the antibody in said dose, once every three weeks, for 1-12 cycles.

In some embodiments, the antibody comprises a V_{H} region comprising an amino acid sequence that is at least 90% identical to the amino acid sequence of SEQ ID NO: 3 and a V_{L} region comprising an amino acid sequence that is at least 90% identical to the amino acid sequence of SEQ ID NO: 4, wherein the V_{H} region comprises three CDRs comprising the amino acid sequences of SEQ ID NOs: 5-7, and the V_{L} region comprises three CDRs comprising the amino acid sequences of SEQ ID NOs: 8-10.

In some embodiments, the antibody comprises a V_{H} region comprising the amino acid sequence of SEQ ID NO: 3 and a variable light chain V_{L} region comprising the amino acid sequence of SEQ ID NO: 4.

In some embodiments, the antibody comprises a heavy chain comprising the amino acid sequence of SEQ ID NO: 1 and a light chain comprising the amino acid sequence of SEQ ID NO: 2.

In some embodiments, the cancer is selected from the group consisting of: gastric cancer, melanoma (e.g., skin cutaneous melanoma), urothelial cancer, lymphoid neoplasm, diffuse large B-cell lymphoma (DLBCL), testicular germ cell tumors (TGCT), mesothelioma, kidney cancer (e.g., kidney renal clear cell carcinoma, kidney renal papillary cell carcinoma, or renal cell carcinoma (RCC)), sarcoma, lung cancer (e.g., lung adenocarcinoma, lung squamous cell carcinoma, and non-small cell lung cancer (NSCLC)) stomach adenocarcinoma, pancreatic adenocarcinoma, head and neck squamous cell carcinoma, ovarian serious cystadenocarcinoma, liver hepatocellular carcinoma, skin cutaneous melanoma, colon adenocarcinoma, breast cancer (e.g., breast invasive carcinoma or triple negative breast cancer), rectum adenocarcinoma, glioblastoma multiforme, uterine corpus endometrial carcinoma, thymoma, bladder cancer, endometrial cancer, Hodgkin's lymphoma, ovarian cancer, anal cancer, biliary cancer, colorectal cancer, and esophageal cancer. In some embodiments, the cancer of the subject is gastric cancer, melanoma, or urothelial cancer.

In some embodiments, the method further comprises administration of one or more additional therapeutic agents to the subject. In some embodiments, the additional therapeutic agent is an anti-cancer agent, e.g., as described herein.

In some embodiments, the subject is a human patient.

The invention also includes compositions (such as those compositions described above and elsewhere herein) for use in the methods described herein.

Other features and advantages of the invention will be apparent from the following detailed description, the drawings, and the claims.

### BRIEF DESCRIPTION OF THE DRAWINGS

FIG. 1 is a series of graphs and a table showing log 2 (LILRB2/IFNg Signature) analysis for non-responders and responders to the following anti-PD1 treatments: gastric cancer (pembrolizumab; anti-PD1), melanoma (nivolumab; anti-PD1), and urothelial cancer (atezolizumab; anti-PD-L1).

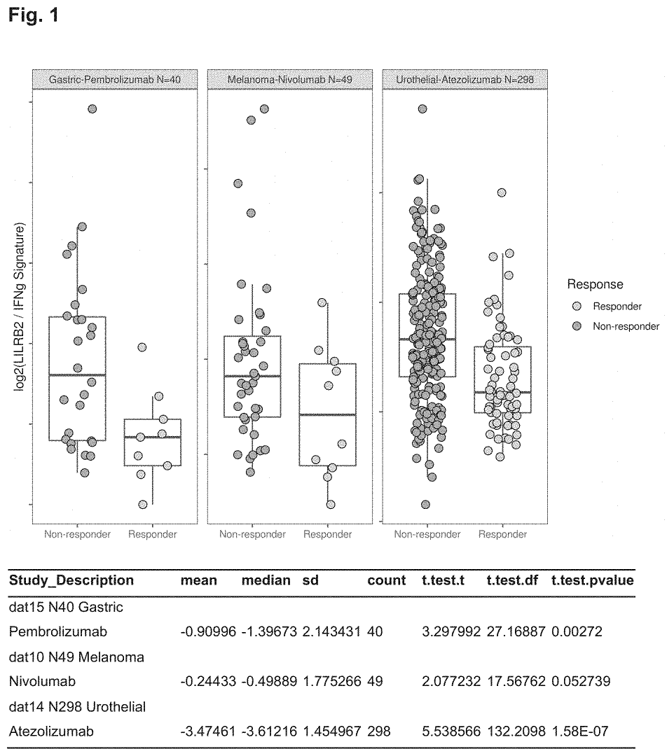

FIG. 2 is a series of graphs and a table showing log 2 (TAM/IFNg Signature) analysis for non-responders and responders to the following anti-PD1 treatments: gastric cancer (pembrolizumab; anti-PD1), melanoma (nivolumab; anti-PD1), and urothelial cancer (atezolizumab; anti-PD-L1).

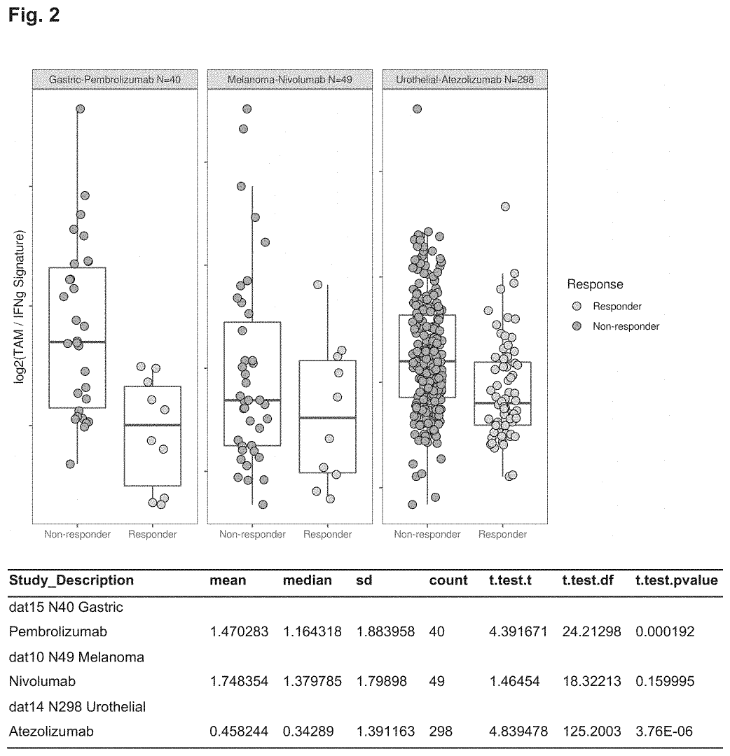

FIG. 3 is a graph showing that TMDD appears to be saturated (parallel elimination) at 300 mg and above.

FIG. 4 is a table showing that mean (SD) clearance and T_{1/2} are stable at 300 mg.

FIG. 5 is a table and a graph showing simulated PK results; 700 mg is selected as the target dose.

FIG. 6 is a series of graphs showing simulated JTX-8064 exposure after the first does and at steady state. The top panel shows simulated concentration ranges after a single dose, and the bottom shows the same after 10 cycles (steady state). The dotted line represents the target dose (median Cmin of 18.3 μg/mL after a single 300 mg dose). The solid black line shows median exposure for each dose, and the shaded blue areas represent the 5^{th} and 95^{th} percentiles of the predicted exposures.

### DETAILED DESCRIPTION

Provided herein are methods of treating subjects having elevated LILRB2 relative to IFNg with a LILRB2 antibody and a PD1 antagonist, as well as methods of treating subjects having a balanced level of LILRB2 and IFNg, or elevated IFNg relative to LILRB2, with a PD1 antagonist, in the absence of a LILRB2 antibody. Also provided are methods of identifying subjects whose cancer may respond to a PD1 antagonist, or a PD1 antagonist in combination with a LILRB2 antibody, as well as related methods for selecting cancer therapies for subjects. Additionally, provided are methods for improving the response of subjects to PD1 antagonist therapy using a LILRB2 antibody. Furthermore, compositions of LILRB2 antibodies in dosage form, as well as therapeutic methods utilizing particular doses of LILRB2 antibodies, are provided. Compositions and kits for use in carrying out the methods described herein are also provided. These and other methods, compositions, and kits of the invention are described further as follows.

The inventions are based, in part, on observations that non-responders to PD1 antagonist therapy have higher LILRB2 or TAM signature to IFNg signature ratios than responders. Also, subjects with lower LILRB2 or TAM signature to IFNg signature ratios are more likely to have complete or partial responses to anti-PD1 therapies. LILRB2 antibodies can be used to suppress immunosuppressive TAMs, leading to increased IFNg production by T cells and improved responses to PD1 antagonist therapy.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

### I. DEFINITIONS

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an,” and “the” includes plural references unless indicated otherwise. Use of the term “or” herein is not meant to imply that alternatives are mutually exclusive. Thus, in this application, the use of “or” means “and/or” unless expressly stated or understood by one skilled in the art. In the context of a multiple dependent claim, the use of “or” refers back to more than one preceding independent or dependent claim.

The terms “nucleic acid molecule,” “nucleic acid,” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an LILRB2 epitope is an antibody that binds this epitope with greater affinity, avidity, more readily, and/or with greater duration than it binds to other LILRB2 epitopes or non-LILRB2 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), e.g., at least 90% pure, at least 95% pure, at least 98% pure, or at least 99% pure.

The term “cross-competes” refers to competitive binding of one molecule with another, e.g., by binding to all or part of the same epitope. Cross-competition can be determined using the experiments described herein (e.g., biolayer interferometry), for example, by detecting no positive response signal upon addition of a second antibody to a sensor after a first antibody is bound to the signal. In particular embodiments, one LILRB2 antibody cross-competes another LILRB2 antibody for binding to LILRB2.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence-based antibody into a mouse, rat, cat, dog, horse, etc. sequence. Antibody fragments also include either orientation of single chain scFvs, tandem di-scFv, diabodies, tandem tri-SdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by the Kabat numbering scheme. Kabat et al., Sequences of Proteins of Immunological Interest, 5th Ed. Public Health Service, National Institutes of Health, Bethesda, Md. (1991). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. CDRs can also be provided as shown in any one or more of the accompanying figures. With the exception of CDR1 in a variable heavy chain region (V_{H}), CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hypervariable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, 1987, J. Mol. Biol. 196:901-917).

The term “heavy chain variable region” or V_{H} as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an Igm antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” of V_{L} as used herein refers to a region comprising at least three light chain CDRs. In some embodiments, the light chain variable region includes the three CDRs and at least FR2 and FR3. In some embodiments, the light chain variable region includes at least light chain LCDR1, framework (FR) 2, LCDR2, FR3, and LCDR3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Nonlimiting exemplary light chain constant regions include λ and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or fewer, 9 or fewer, 8 or fewer, 7 or fewer, 6 or fewer, 5 or fewer, 4 or fewer, 3 or fewer, or 2 or fewer. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the equilibrium dissociation constant (K_{D}). Affinity can be measured by common methods known in the art (such as, for example, ELISA K_{D}, KinExA, bio-layer interferometry (BLI), and/or surface plasmon resonance devices (such as a BIACORE® device), including those described herein).

The term “K_{D}”, as used herein, refers to the equilibrium dissociation constant of an antibody-antigen interaction.

In some embodiments, the “K_{D}” of the antibody is measured by using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM), before injection at a flow rate of 5 μL/minute to achieve approximately 10 response units (RU) of coupled protein. Following the injection of antigen, 1 M ethanolamine is injected to block unreacted groups. For kinetics measurements, serial dilutions of polypeptide, for example, full length antibody, are injected in PBS with 0.05% TWEEN-20™ surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., 1999, J. Mol. Biol. 293:865-881. If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In some embodiments, the difference between said two values (for example, K_{D} values) is substantially the same, for example, less than about 50%, less than about 40%, less than about 30%, less than about 20%, and/or less than about 10% as a function of the reference/comparator value.

In some embodiments, the difference between said two values (for example, K_{D} values) is substantially different, for example, greater than about 10%, greater than about 20%, greater than about 30%, greater than about 40%, and/or greater than about 50% as a function of the value for the reference/comparator molecule.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al., 1993, Ann. Biol. Clin. 51:19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is FORTEBIO® OCTET® RED96 system (Pall Corporation). See, e.g., Abdiche et al., 2008, Anal. Biochem. 377: 209-277.

The term “kₒₙ”, as used herein, refers to the rate constant for association of an antibody to an antigen. Specifically, the rate constants (kₒₙ and k_{off}) and equilibrium dissociation constant (K_{D}) are measured using IgGs (bivalent) with monovalent antigen (e.g., LILRB2 antigen). “Kₒₙ”, “kₒₙ”, “association rate constant”, or “ka”, are used interchangeably herein. The value indicates the binding rate of a binding protein to its target antigen or the rate of complex formation between an antibody and antigen, shown by the equation:

Antibody(“Ab”)+Antigen(“Ag”)→Ab−Ag.

The term “k_{off}”, as used herein, refers to the rate constant for dissociation of an antibody from the antibody/antigen complex. k_{off} is also denoted as “K_{off}” or the “dissociation rate constant”. This value indicates the dissociation rate of an antibody from its target antigen or separation of Ab-Ag complex over time into free antibody and antigen as shown by the equation:

Ab+Ag←Ab−Ag.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing maturation or activation (e.g., myeloid cell maturation or activation), inhibiting maturation or activation (e.g., myeloid cell maturation or activation), inducing cytokine expression or secretion (e.g., inflammatory cytokines or immunosuppressive cytokines), inducing apoptosis, and enzymatic activity. In some embodiments, biological activity of an LILRB2 protein includes, for example, conversion of M2-like macrophages to M1-like macrophages.

An “M2-like macrophage,” as used herein, refers to a macrophage characterized by one or more immunosuppressive characteristics, relative to a reference. Immunosuppressive characteristics include decreased maturation marker or activation marker expression (e.g., decreased expression of one or more costimulatory markers (e.g., CD80 or CD86), decreased antigen presentation (e.g., by HLA expression), decreased expression of inflammatory cytokines (e.g., TNFα, IL-6, or IL-1β), and increased regulatory or suppressive marker expression (e.g., increased IL-10 or CCL-2 expression or secretion). Immunosuppressive characteristics may additionally or alternatively be characterized by decrease in immunogenic or inflammatory gene expression, or increase in immunosuppressive or immunoregulatory gene expression, according to methods known in the art. Immunosuppressive characteristics may additionally or alternatively be characterized by one or more functional qualities, such as the ability to inhibit activation and/or expansion of other immune cells. Assays suitable for identifying a macrophage as an M2-like macrophage are known in the art and described herein. For example, a primary human macrophage assay can be used to determine whether a macrophage is an M2-like macrophage or an M1-like macrophage. In some instances, an M2-like macrophage is a tumor-associated macrophage. In the context of determining whether a macrophage is an M2-like macrophage, a reference can be provided by a control macrophage of the same or different origin (e.g., an untreated control or an LPS-treated control). In embodiments in which a candidate macrophage is a tumor-associated macrophage, a control may be a non-tumor-associated macrophage (e.g., from a healthy donor). Alternatively, a reference can be a predetermined threshold, e.g., a parameter derived from an art-known immunosuppressive threshold.

An “M1-like macrophage,” as used herein, refers to a macrophage characterized by one or more immunogenic (e.g., immunostimulatory or activatory) characteristics, relative to a reference. Immunogenic characteristics include increased maturation marker or activation marker expression (e.g., increased expression of one or more costimulatory markers (e.g., CD80 or CD86), increased antigen presentation (e.g., by HLA expression), increased expression of activating cytokines (e.g., TNFα, IL-6, or IL-1β), decreased regulatory or suppressive marker expression (e.g., decreased IL-10 or CCL-2 expression or secretion). Immunogenic characteristics may additionally or alternatively be characterized by increase in immunogenic or inflammatory gene expression, or decrease in immunosuppressive or immunoregulatory gene expression, according to methods known in the art. Immunogenic characteristics may additionally or alternatively be characterized by one or more functional qualities, such as the ability to activate and/or expand other immune cells. Assays suitable for identifying a macrophage as an M1-like macrophage are known in the art and described herein. For example, a primary human macrophage assay can be used to determine whether a macrophage is an M2-like macrophage or an M1-like macrophage. In some instances, an M1-like macrophage is a tumor-associated macrophage (e.g., a tumor-associated macrophage that has been exposed to an antibody to LILRB2). In the context of determining whether a macrophage is an M1-like macrophage, a reference can be provided by a control macrophage of the same or different origin (e.g., an untreated control or an immunosuppressed control). In embodiments in which a candidate macrophage is a tumor-associated macrophage, a control may be a non-tumor-associated macrophage (e.g., from a healthy donor). Alternatively, a reference can be a predetermined threshold, e.g., a parameter derived from an art-known immunogenic threshold.

“Conversion of an M2-like macrophage to an M1-like macrophage” can be identified upon detection of an increase in any one or more characteristics of an M1-like macrophage, a decrease in any one or more characteristics of an M2-like macrophage, or any combination thereof.

As used herein, a “human monocyte-derived macrophage,” a “human monocyte-differentiated macrophage,” or an “HMDM” refers to a macrophage that has been derived from a primary human monocyte. In some embodiments, the primary human macrophage is derived from monocytes from whole blood (e.g., from a PBMC population). In some embodiments, primary human monocytes are incubated in the presence of M-CSF for seven days. A human monocyte-derived macrophage can be obtained using the methods described in Example 6.

As used herein, the term “tetramer blocking assay” refers to an assay including the following steps:
(1) plate 1×10⁵ macrophages (e.g., human monocyte differentiated macrophages (HMDMs)) in a well of a 96-well round-bottom tissue culture plate;
(2) add 50 μL test antibody (e.g., LILRB2 antibody or isotype control) in buffer (e.g., FACS buffer (1×DPBS containing 2% HI-FBS (Sigma)+0.05% Sodium Azide));
(3) incubate 30 minutes at 4° C.;
(4) wash cells in buffer (e.g., FACS buffer) and resuspend in 50 μL buffer (e.g., FACS buffer) containing 1 μg/mL tetramer (e.g., fluorochrome-labeled tetramer, e.g., HLA-G or HLA-A2 tetramer);
(5) incubate protected from light for 30-60 minutes at 4° C.;
(6) wash cells in buffer (e.g., FACS buffer); and
(7) quantify tetramer binding (e.g., using flow cytometry).

A “chimeric antibody” as used herein refers to an antibody in which a portion of the heavy and/or light chain is derived from a particular source or species, while at least a part of the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species. The chimeric construct can also be a functional fragment, as noted above.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody.

As will be appreciated, a humanized sequence can be identified by its primary sequence and does not necessarily denote the process by which the antibody was created.

An “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein encompasses antibodies produced in humans, antibodies produced in non-human animals that comprise human immunoglobulin genes, such as XENOMOUSE® mice, and antibodies selected using in vitro methods, such as phage display (Vaughan et al., 1996, Nat. Biotechnol., 14:309-314; Sheets et al., 1998, Proc. Natl. Acad. Sci. (USA), 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

A “functional Fc region” possesses an “effector function” of a native sequence Fc region. Exemplary “effector functions” include Fc receptor binding; C1q binding; CDC; ADCC; phagocytosis; down regulation of cell surface receptors (for example B cell receptor; BCR), etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. See, for example, Daeron, 1997, Annu. Rev. Immunol. 15:203-234. FcRs are reviewed, for example, in Ravetch and Kinet, 1991, Annu. Rev. Immunol 9:457-92; Capel et al., 1994, Immunomethods, 4:25-34; and de Haas et al., 1995, J. Lab. Clin. Med. 126:330-41. Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., 1976, J. Immunol. 117:587 and Kim et al., 1994, J. Immunol. 24:249) and regulation of homeostasis of immunoglobulins. Methods of measuring binding to FcRn are known. See, for example, Ghetie and Ward, 1997, Immunol. Today, 18(12):592-598; Ghetie et al., 1997, Nat. Biotechnol., 15(7):637-640; Hinton et al., 2004, J. Biol. Chem. 279(8):6213-6216; and WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (for example B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, 1991, Annu. Rev. Immunol 9:457-92. To assess ADCC activity of a molecule of interest, an in vitro ADCC assay, such as that described in U.S. Pat. Nos. 5,500,362, 5,821,337, or 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al., 1998, Proc. Natl. Acad. Sci. (USA) 95:652-656. Additional polypeptide variants with altered Fc region amino acid sequences (polypeptides with a variant Fc region) and increased or decreased ADCC activity are described, for example, in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, for example, as described in Gazzano-Santoro et al., 1996, J. Immunol. Methods 202:163, may be performed. Polypeptide variants with altered Fc region amino acid sequences (polypeptides with a variant Fc region) and increased or decreased C1q binding capability are described, for example, in U.S. Pat. No. 6,194,551 B1, U.S. Pat. Nos. 7,923,538, 7,994,290, and WO 1999/51642. See also, for example, Idusogie et al., 2000, J. Immunol. 164: 4178-4184.

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, for example, 0-20% binding to the FcR compared to a native sequence IgG Fc region.

The polypeptide variant which “mediates antibody-dependent cell-mediated cytotoxicity (ADCC) in the presence of human effector cells more effectively” than a parent antibody is one which in vitro or in vivo is more effective at mediating ADCC, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

The phrase “substantially increased” or “elevated” as used herein, denotes a sufficiently high degree of increase between a numeric value and a reference numeric value such that one of skill in the art would consider the difference between the two values to be of statistical significance within the context of the biological characteristic measured by said values. In some embodiments, the substantially increased numeric values is increased by greater than about any one of 10%, 20%, 25%, 30%, 35%, 40%, 45%, 50%, 60%, 70%, 80%, 90%, or 100% compared to the reference value.

The term “elevated” when used in the context of a ratio between LILRB2 and IFNg, as described herein (e.g., LILRB vs. IFNg per se or IFNg signature, or TAM signature vs. IFNg per se or IFNg signature), can be considered to be a level that is above a reference level, wherein the reference level is obtained by: 1. identifying a population of patients treated with a PD-1 inhibitor, 2. dividing the population of patients into a responding group and non-responding group 3. determining ratio of LILRB2 and IFNg (as described herein) for each group (considering both the mean and standard deviations), and 4. optionally determining statistical significance using t-test or another appropriate test, 5. where the average ratio of the non-responding group is the reference level.

The term “leader sequence” refers to a sequence of amino acid residues located at the N-terminus of a polypeptide that facilitates secretion of a polypeptide from a mammalian cell. A leader sequence can be cleaved upon export of the polypeptide from the mammalian cell, forming a mature protein. Leader sequences can be natural or synthetic, and they can be heterologous or homologous to the protein to which they are attached.

A “native sequence” polypeptide comprises a polypeptide having the same amino acid sequence as a polypeptide found in nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (for example, an extracellular domain sequence), naturally occurring variant forms (for example, alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual,” “patient,” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example, based on physical, biochemical, chemical, and/or physiological characteristics. For example, the phrase “test sample,” and variations thereof, refers to any sample obtained from a subject of interest that would be expected or is known to contain a cellular and/or molecular entity that is to be characterized. By “tissue or cell sample” is meant a collection of similar cells obtained from a tissue of a subject or patient. The source of the tissue or cell sample may be a tumor sample, blood (e.g., peripheral blood) or any blood constituents; solid tissue as from a fresh, frozen, and/or preserved organ or tissue sample or biopsy or aspirate; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. In some embodiments, a sample includes peripheral blood obtained from a subject or patient, which includes CD4+ cells. In some embodiments, a sample includes CD4+ cells isolated from peripheral blood. In some embodiments, a sample is a sample of peripheral blood mononuclear cells (PBMCs). A “sample” or “patient sample” includes, for example, a “test sample,” a “tissue sample,” or a “cell sample.”

A “control,” “control sample,” “reference,” or “reference sample” as used herein, refers to any sample, standard, or level that is used for comparison purposes. A control or reference may be obtained from a healthy and/or non-diseased sample. In some examples, a control or reference may be obtained from an untreated sample or patient. In some examples, a reference is obtained from a non-diseased or non-treated sample of a subject individual. In some examples, a reference is obtained from one or more healthy individuals who are not the subject or patient. In some embodiments, a control sample, reference sample, reference cell, or reference tissue is obtained from the patient or subject at a time point prior to one or more administrations of a treatment (e.g., one or more anti-cancer treatments), or prior to being subjected to any of the methods of the invention.

A “disease” or “disorder” as used herein refers to a condition where treatment is needed and/or desired. In some embodiments, the disease or disorder is cancer.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include kidney cancer (e.g., renal cell carcinoma, e.g., papillary renal cell carcinoma), squamous cell cancer, mesothelioma, teratoma, small-cell lung cancer, pituitary cancer, esophageal cancer, astrocytoma, soft tissue sarcoma, lung cancer (e.g., non-small cell lung cancer, adenocarcinoma of the lung, squamous carcinoma of the lung), cancer of the peritoneum, hepatocellular cancer, gastrointestinal cancer (e.g., stomach cancer), pancreatic cancer, cervical cancer, ovarian cancer, liver cancer, bladder cancer, hepatoma, breast cancer, colon cancer, colorectal cancer, rectal cancer, endometrial or uterine carcinoma, salivary gland carcinoma, liver cancer, prostate cancer, vulval cancer, thyroid cancer, thymoma, hepatic carcinoma, brain cancer, glioma, glioblastoma, endometrial cancer, testis cancer, cholangiocarcinoma, cholangiosarcoma, gallbladder carcinoma, gastric cancer, melanoma (e.g., uveal melanoma), pheochromocytoma, paraganglioma, adenoid cystic carcinoma, and various types of head and neck cancer (e.g., squamous head and neck cancer). These cancers, and others, can be treated or analyzed according to the methods of the invention.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering an anti-LILRB2 antibody. “Ameliorating” also includes shortening or reduction in duration of a symptom.

In the context of cancer, the term “treating” includes any or all of: inhibiting growth of cancer cells, inhibiting replication of cancer cells, lessening of overall tumor burden and ameliorating one or more symptoms associated with the disease.

The term “biological sample” means a quantity of a substance from a living thing or formerly living thing. Such substances include, but are not limited to tumor biopsies, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” refers to a composition known to not contain an analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (for example, analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (for example, severity of disease, progression, non-progression, improvement, etc.). While the present disclosure may provide exemplary predetermined levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased on non-treated sample of a subject individual. In some examples, a reference is obtained from one or more healthy individuals who are not the subject or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce,” “inhibit,” or “prevent” do not denote or require complete prevention over all time.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result. The therapeutically effective amount of the treatment of the invention can be measured by various endpoints commonly used in evaluating cancer treatments, including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

A “PD-1 therapy” encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies. Nonlimiting exemplary PD-1 therapies include nivolumab (BMS-936558, MDX-1106, ONO-4538); pidilizumab, lambrolizumab/pembrolizumab (KEYTRUDA®, MK-3475); durvalumab; RG-7446; MSB-0010718C; AMP-224; BMS-936559 (an anti-PD-L1 antibody); AMP-514; MDX-1105; ANB-011; anti-LAG-3/PD-1; anti-PD-1 Ab (CoStim); anti-PD-1 Ab (Kadmon Pharm.); anti-PD-1 Ab (Immunovo); anti-TIM-3/PD-1 Ab (AnaptysBio); anti-PD-L1 Ab (CoStim/Novartis); MEDI-4736 (an anti-PD-L1 antibody, Medimmune/AstraZeneca); RG7446/MPDL3280A (an anti-PD-L1 antibody, Genentech/Roche); KD-033, PD-1 antagonist (Agenus); STI-A1010; STI-A1110; TSR-042; and other antibodies that are directed against programmed death-1 (PD-1) or programmed death ligand 1 (PD-L1).

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s) or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “conjugate” refers to an antibody that is chemically linked to a second chemical moiety, such as a therapeutic or cytotoxic agent. The term “agent” includes a chemical compound, a mixture of chemical compounds, a biological macromolecule, or an extract made from biological materials. In some embodiments, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

As used herein, the term “flow cytometry” generally refers to a technique for characterizing biological particles, such as whole cells or cellular constituents, by flow cytometry. Methods for performing flow cytometry on samples of immune cells are well known in the art (see e.g., Jaroszeski et al., Method in Molecular Biology (1998), vol. 91: Flow Cytometry Protocols, Humana Press; Longobanti Givan, (1992) Flow Cytometry, First Principles, Wiley Liss). All known forms of flow cytometry are intended to be included, particularly fluorescence activated cell sorting (FACS), in which fluorescent labeled molecules are evaluated by flow cytometry.

The term “amplification” refers to the process of producing one or more copies of a nucleic acid sequence or its complement. Amplification may be linear or exponential (e.g., PCR).

The technique of “polymerase chain reaction” or “PCR” as used herein generally refers to a procedure wherein a specific region of nucleic acid, such as RNA and/or DNA, is amplified as described, for example, in U.S. Pat. No. 4,683,195. Generally, oligonucleotide primers are designed to hybridize to opposite strands of the template to be amplified, a desired distance apart. PCR can be used to amplify specific RNA sequences, specific DNA sequences from total genomic DNA, and cDNA transcribed from total cellular RNA, bacteriophage or plasmid sequences, etc.

“Quantitative real time PCR” or “qRT-PCR” refers to a form of PCR wherein the PCR is performed such that the amounts, or relative amounts of the amplified product can be quantified. This technique has been described in various publications including Cronin et al., Am. J. Pathol. 164(1):35-42 (2004); and Ma et al., Cancer Cell 5:607-616 (2004).

The term “target sequence,” “target nucleic acid,” or “target nucleic acid sequence” refers generally to a polynucleotide sequence of interest, e.g., a polynucleotide sequence that is targeted for amplification using, for example, qRT-PCR.

The term “detection” includes any means of detecting, including direct and indirect detection.

### II. DETERMINATION OF EXPRESSION LEVELS

In some embodiments, the methods of the invention include determining RNA levels (e.g., RNA signature scores) of a sample of a cancer of a subject (e.g., a human patient). Accordingly, RNA levels (e.g., an RNA signature score) can be determined in a sample of tissue including cancer (e.g., a tumor sample, a blood sample, or a tissue sample wherein the tissue comprises cancer cells) that is obtained from a subject. Alternatively, protein levels can be determined in such samples.

a. Sample Preparation

Cancers that can be analyzed according to the present invention can optionally be primary, metastatic, or recurrent, and may be of any type (e.g., as listed elsewhere herein). Furthermore, the cancers may be of any stage including, e.g., Stage I, II, III, or IV, and/or of any histology. A subject (e.g., a human patient) having the cancer can be of any age or gender and may have any treatment history and/or extent or duration of disease or remission.

The cancer sample can be obtained using a variety of different procedures that are selected based on factors including, for example, the type, location, and size of the cancer. Exemplary methods include tissue biopsy, e.g., needle biopsy (e.g., fine needle aspiration, core needle biopsy, and image-guided biopsy); surgical biopsy (e.g., incisional biopsy or excisional biopsy); liquid biopsy (e.g., by obtaining circulating tumor cells); endoscopic biopsy; and scrape or brush biopsy.

Samples are processed for detection of RNA or protein using standard methods, which are selected based on, e.g., the type of cancer and the assay format to be used. In certain embodiments, the tumor tissue can be micro-dissected from the remaining sample prior to isolation or detection of RNA or protein. For example, the samples can be fixed using, e.g., neutral buffered formalin, glutaraldehyde, or paraformaldehyde. In some examples, a tissue sample fixed in formalin is also embedded in paraffin to prepare a formalin-fixed paraffin-embedded (or FFPE) tissue sample. The tissue sample can be sectioned and assayed as a fresh specimen. Alternatively, the tissue sample can be frozen for further processing, e.g., sectioning or nucleic acid extraction. In other examples, the samples can be in the form of a tissue or cell extract, or can be in the form of isolated, individual cells. Accordingly, the sample can be a solid tissue sample or slice, fluid derived from a tumor, fraction, or extract, lymph tissue, blood, or other tissue comprising the cancer cells. Moreover, the cancer cells can be cultured, washed, or otherwise selected to remove non-cancerous cells from the sample, and optionally the cancer cells can be sorted by fluorescence activated cell sorting or other cell sorting technique. Detection can be carried out on tissues, cells, tissue extracts, cell extracts, whole cell lysates, protein extracts, and nucleic acid extracts (e.g., RNA extracts). With respect to the latter, RNA can be extracted from cells using standard RNA extraction techniques and kits including, for example, acid guanidinium-acid-phenol extraction (TRIzol and TRI reagent), phenol/guanidine isothiocyanate extraction (RNAzolB Biogenesis), silica technology and glass fiber filters (e.g. RNeasy RNA preparation kits (Qiagen)), magnetic bead technology (e.g., dynabeads mRNA DIRECT micro), lithium chloride and urea isolation, oligo(dt)-cellulose column chromatography, and non-column poly (A)+ purification.

b. Detection Methods

RNA, including, e.g., the components of an RNA signature, can be detected in samples by analysis of transcribed polynucleotides, variants, portions, or reverse transcripts thereof (e.g., pre-mRNA, mRNA, splice variants, or cDNA). RNA detection methods that can be used include, e.g., nucleic acid sequence based amplification (NASBA) combined with molecular beacon detection molecules (Compton, Nature 350(6313):91-92, 1991), a flap endonuclease-based assay (e.g., Invader™, Third Wave Technologies), direct mRNA capture with branched DNA (QuanitGene™, Panomics) or Hybrid Capture™ (Digene), RNA-seq, RT-PCR, quantitative PCR, microarray analysis, ligase chain reaction, RNAse protection, nuclease protection combined with array detection (e.g., ArrayPlate™, HTG Molecular, Tucson, Ariz.; Martel et al., Assay and Drug Dev. Tech. 1(1):61-72, 2002), northern blotting, and nuclear run-on assays. Other approaches include hybridization-based methods employing a capture probe and a reporter probe, wherein the capture probe includes a sequence coupled to an immobilization tag for immobilization of a complex including the capture probe, analyte, and detection probe for analysis (e.g., a NanoString® system, such as the nCounter® Analysis System; NanoString® Technologies, Seattle, Wash.). Alternatively, RNA can be analyzed by hybridization of tissue samples with labeled probes. Additional details concerning exemplary methods that can be used to detect RNA are provided below.

In some embodiments, the methods provided herein include measuring an RNA (e.g., mRNA) level. In some embodiments, the methods provided herein include measuring an RNA signature, e.g., a plurality of RNA levels that are predictive of or correlated to improved responses to combined LILRB2 antibody and PD-1 antagonist therapy, or PD-1 antagonist therapy in the absence of a LILRB2 antibody, as described herein. In some embodiments, a single species of RNA is detected (e.g., RNA encoding LILRB2). In some embodiments, an RNA signature is detected (e.g., a TAM and/or IFNg RNA signature). In some embodiments, the RNA signature includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty eight, at least twenty-nine, at least thirty, at least thirty-one, at least thirty-two, at least thirty-three, at least thirty-four, at least thirty-five, at least thirty-six, at least thirty-seven, at least thirty-eight, at least thirty-nine, at least forty, at least forty-one, at least forty-two, at least forty-three, or at least forty-four RNA levels, the RNA levels being levels of RNAs selected from LILRB2, Table 2 (TAM), or Table 3 (IFNg).

Any suitable method of determining RNA (e.g., mRNA) levels may be used. Methods for the evaluation of RNA include, for example, hybridization assays using complementary nucleic acid probes (such as in situ hybridization using labeled riboprobes specific for target sequences, Northern blot, and related techniques), various nucleic acid amplification assays (such as RT-PCR using complementary primers specific for target sequences and other amplification type detection methods, such as, for example, branched DNA, SISB A, TMA and the like), and sequencing-based assays (e.g., RNA-seq).

Accordingly, in some embodiments, the RNA (e.g., mRNA) level is determined by the use of Nanostring technologies.

In some embodiments, the RNA (e.g., mRNA) level is determined by quantitative RT-PCR. In some embodiments, the mRNA level is determined by digital PCR. In some embodiments, the mRNA level is determined by RNA-Seq. In some embodiments, the mRNA level is determined by RNase Protection Assay (RPA). In some embodiments, the mRNA level is determined by Northern blot. In some embodiments, the mRNA level is determined by in situ hybridization (ISH). In some embodiments, the mRNA level is determined by a method selected from quantitative RT-PCR, microarray, digital PCR, rNA-Seq, RNase Protection Assay (RPA), Northern blot, and in situ hybridization (ISH).

RNA-seq is a technique based on enumeration of RNA transcripts using next-generation sequencing methodologies. The level of an mRNA is determined using the frequency of observation of fragments of its sequence (see, Wang et al., Nat. Rev. Genet. 10:57-63, 2009).

Northern blotting involves the use of electrophoresis to separate RNA samples by size, and detection with hybridization probes complementary to part of or the entire target sequence (see, e.g., Trayhurn, Northern Blotting. Pro. Nutrition Soc. 55:583-589, 1996).

Quantitative RT-PCR involves reverse-transcribing mRNA and then amplifying the resulting cDNA by polymerase chain reaction (PCR), which can be monitored in real time, e.g., by measuring fluorescence, wherein dye signal is a readout of the amount of product. The dye can be, e.g., an intercalating dye, or a dye attached to a probe also including a quencher, wherein degradation of the probe releases the dye and results in fluorescence, the degradation being catalyzed by an exonuclease activity driven by product formation, as in the TaqMan® assay. In some embodiments, a method for detecting a target mRNA in a biological sample includes producing cDNA from the sample by reverse transcription using at least one primer; amplifying the resulting cDNA using a target polynucleotide as sense and antisense primers to amplify target cDNAs therein; and detecting the presence of the amplified target cDNA. In addition, such methods can include one or more steps that allow one to determine the levels of target mRNA in a biological sample (e.g., by simultaneously examining the levels a reference mRNA sequence, e.g., a “housekeeping” gene such as the reference RNAs described herein). Optionally, the sequence of the amplified target cDNA can be determined.

In digital PCR, a sample is partitioned into a plurality of reaction areas and PCR is conducted in the areas. The number of areas that are positive, i.e., in which detectable product formation occurs, can be used to determine the level of the target sequence in the original sample.

In an RPA, a sample is contacted with a probe under hybridization conditions and then with a single-stranded RNA nuclease. Formation of double-stranded complexes of probe with target protect the probe from degradation, such that the amount of probe remaining can be used to determine the level of the target.

In ISH, a cell or tissue sample is contacted with a probe that hybridizes to a target RNA and hybridization is detected to determine the level of the target.

In some embodiments, the methods include protocols in which mRNAs, such as target mRNAs, are detected in a tissue or cell sample, or RNA extracted therefrom, using microarrays. In these assays, test and control mRNA samples from test and control tissue or cell samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene. Microarray technology utilizes nucleic acid hybridization techniques and computing technology to evaluate the mRNA expression profile of thousands of genes within a single experiment (see, e.g., WO 01/75166; U.S. Pat. Nos. 5,700,637; 5,445,934; 5,807,522; Lockart, Nature Biotechnology 14:1675-1680, 1996; Cheung et al., Nature Genetics 21(Suppl):15-19, 1999). DNA microarrays are miniature arrays containing gene fragments that are either synthesized directly onto or spotted onto glass or other substrates. Thousands of genes are usually represented in a single array. A typical microarray experiment can involve the following steps: 1) preparation of fluorescently labeled target from RNA isolated from the sample, 2) hybridization of the labeled target to the microarray, 3) washing, staining, and scanning of the array, 4) analysis of the scanned image, and 5) generation of gene expression profiles. Two types of DNA microarrays are oligonucleotide (usually 25 to 70 mers) arrays and gene expression arrays containing PCR products prepared from cDNAs. In forming an array, oligonucleotides can be either prefabricated and spotted to the surface or directly synthesized onto the surface (in situ). In some embodiments, a DNA microarray is a single-nucleotide polymorphism (SNP) microarray, e.g., Affymetrix® SNP Array 6.0.

The Affymetrix GeneChip® system is a commercially available microarray system that comprises arrays fabricated by direct synthesis of oligonucleotides on a glass surface. In probe/gene arrays, oligonucleotides, usually 25 mers, are directly synthesized onto a glass wafer by a combination of semiconductor-based photolithography and solid phase chemical synthesis technologies. Each array contains up to 400,000 different oligos and each oligo is present in millions of copies. Since oligonucleotide probes are synthesized in known locations on the array, the hybridization patterns and signal intensities can be interpreted in terms of gene identity and relative levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligo. The Microarray Suite software subtracts the hybridization intensities of the mismatch probes from those of the perfect match probes to determine the absolute or specific intensity value for each probe set. Probes are chosen based on current information from Genbank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

In some embodiments, the level of at least one mRNA is normalized. In some embodiments, the levels of at least two mRNAs are normalized and compared to each other. In some embodiments, such normalization may allow comparison of mRNA levels when the levels are not determined simultaneously and/or in the same assay reaction. One skilled in the art can select a suitable basis for normalization, such as at least one reference mRNA or other factor, depending on the assay. In some embodiments, at least one reference mRNA comprises a housekeeping gene. In some embodiments, at least one reference mRNA includes two or more, three or more, four or more, five or more, six or more, seven or more, eight or more, nine or more, or ten or more normalization genes (e.g., housekeeping genes), or any ranges between these values.

Expression of the biomarkers can also be analyzed at the protein level using, e.g., immunological based methods (e.g., immunohistochemistry (IHC), ELISA, FACS, capillary electrophoresis, HPLC, TLC, RIA, Western blotting, immunofluorescence, and proteomic methods (e.g., mass spectrometry). In some embodiments, the methods provided herein include measuring an expression signature, e.g., a plurality of protein levels that are predictive of or correlated to improved responses to combined LILRB2 antibody and PD-1 antagonist therapy, or PD-1 antagonist therapy in the absence of a LILRB2 antibody, as described herein. In some embodiments, a single species of protein is detected (e.g., LILRB2). In some embodiments, an expression signature is detected (e.g., a TAM and/or IFNg expression signature; see Tables 2 and 3, respectively). In some embodiments, the expression signature includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty eight, at least twenty-nine, at least thirty, at least thirty-one, at least thirty-two, at least thirty-three, at least thirty-four, at least thirty-five, at least thirty-six, at least thirty-seven, at least thirty-eight, at least thirty-nine, at least forty, at least forty-one, at least forty-two, at least forty-three, or at least forty-four protein levels, the protein levels being protein levels selected from LILRB2, Table 2 (TAM), or Table 3 (IFNg).

c. RNA Signature Score Determination

In some embodiments, an RNA or gene signature score for each sample is determined as follows. Gene expression data is analyzed in TPM (transcripts per million) or some other gene expression value (e.g., FPKM, RPKM, or delta CT); reference will be to TPM, as just an example, for the remainder of this section. Signatures are calculated per sample by gathering the gene expression values for all genes in the signature and taking the geometric mean of these values, optionally plus an arbitrary value (e.g., 0.01, 0.001, or 0.0001) should there be a gene having a value of zero and it is not desired to remove any samples with such a value:
Signature=geometric mean (x+0.0001)
X is equal to the values of all genes comprised in the gene signature
The 0.0001 is an additional, arbitrary factor that allows for the calculation of signature scores in the event that one of the genes has zero TPM, as noted above.

Values of individual genes are TPM and graphed in a log scale, e.g., log 2(TPM) or log 10(TPM). Ratio of LILRB2/IFNG signature is taken by dividing LILRB2 by IFNG signature and converting the ratio to log scale (e.g., log 2 or log 10 scale): Log 2(LILRB2/IFNG Signature) or Log 10(LILRB2/IFNG Signature). Ratio of TAM signature/IFNG signature is taken by dividing TAM signature by IFNG signature and converting the ratio to log scale (e.g., log 2 or log 10 scale): Log 2(TAM Signature/IFNG Signature) or Log 10(TAM Signature/IFNG Signature).

It is understood that the methods described above are exemplary only and that other approaches can be used to obtain information that is equivalent to that set forth above but is expressed differently.

d. Kits and Compositions

Provided herein are also polynucleotides, kits, medicines, and compositions suitable for use in methods such as those described herein.

In some embodiments, a polynucleotide provided herein is isolated. In some embodiments, a polynucleotide provided herein is detectably labeled, e.g., with a radioisotope, a fluorescent agent, or a chromogenic agent. In another embodiment, a polynucleotide is a primer. In another embodiment, a polynucleotide is an oligonucleotide, e.g., an mRNA-specific oligonucleotide. In another embodiment, an oligonucleotide may be, for example, from 7-60 nucleotides in length, 9-45 nucleotides in length, 15-30 nucleotides in length, or 18-25 nucleotides in length. In another embodiment, an oligonucleotide may be, e.g., PNA, morpholino-phosphoramidates, LNA, or 2′-alkoxyalkoxy. Polynucleotides as provided herein are useful, e.g., for the detection of target sequences, such as sequences contained within the RNA signature or a reference mRNA, such as the reference mRNAs discussed above.

Detection can involve hybridization, amplification, and/or sequencing, as discussed above.

In some embodiments, compositions are provided that include a plurality of polynucleotides, the plurality including at least a first polynucleotide specific for a first mRNA and a second polynucleotide specific for a second mRNA, the first and second mRNAs being selected from the mRNAs in the RNA signature. In some embodiments, the plurality further comprises a third polynucleotide specific for a third mRNA, the third mRNA being selected from the mRNAs in the RNA signature. In some embodiments, the plurality further comprises a fourth polynucleotide specific for a fourth mRNA, the fourth mRNA being selected from the mRNAs in the RNA signature. In some embodiments, the plurality further comprises a fifth polynucleotide specific for a fifth mRNA, the fifth mRNA being selected from the mRNAs in the RNA signature. In some embodiments, the plurality further comprises a sixth polynucleotide specific for a sixth mRNA, the sixth mRNA being selected from the mRNAs in the RNA signature. In some embodiments, the plurality further comprises a seventh polynucleotide specific for a seventh mRNA, the seventh mRNA being selected from the mRNAs in the RNA signature. In some embodiments, the plurality further comprises an eighth polynucleotide specific for an eighth mRNA, the eighth mRNA being selected from the mRNAs in the RNA signature. In some embodiments, the plurality further comprises a ninth polynucleotide specific for a ninth mRNA, the ninth mRNA being selected from the mRNAs in the RNA signature. In some embodiments, the plurality further comprises a tenth polynucleotide specific for a tenth mRNA, the tenth mRNA being selected from the mRNAs in the RNA signature. In some embodiments, the plurality further comprises an eleventh polynucleotide specific for an eleventh mRNA, the eleventh mRNA being selected from the mRNAs in the RNA signature. In some embodiments, the plurality further comprises a twelfth polynucleotide specific for a twelfth mRNA, the twelfth mRNA being selected from the mRNAs in the RNA signature. In some embodiments, the plurality further comprises a thirteenth polynucleotide specific for a thirteenth mRNA, the thirteenth mRNA being selected from the mRNAs in the RNA signature. In some embodiments, the plurality further comprises a fourteenth polynucleotide specific for a fourteenth mRNA, the fourteenth mRNA being selected from the mRNAs in the RNA signature. In some embodiments, the plurality further comprises a fifteenth polynucleotide specific for a fifteenth mRNA, the fifteenth mRNA being selected from the mRNAs in the RNA signature. In some embodiments, the plurality further comprises a sixteenth polynucleotide specific for a sixteenth mRNA, the sixteenth mRNA being selected from the mRNAs in the RNA signature. In some embodiments, the plurality further comprises a seventeenth polynucleotide specific for a seventeenth mRNA, the seventeenth mRNA being selected from the mRNAs in the RNA signature. In some embodiments, the plurality further comprises an eighteenth polynucleotide specific for an eighteenth mRNA, the eighteenth mRNA being selected from the mRNAs in the RNA signature. In some embodiments, the plurality further comprises a nineteenth polynucleotide specific for a nineteenth mRNA, the nineteenth mRNA being selected from the mRNAs in the RNA signature. In some embodiments, the plurality further comprises an eighteenth polynucleotide specific for an eighteenth mRNA, the eighteenth mRNA being selected from the mRNAs in the RNA signature. In some embodiments, the plurality further comprises a twentieth polynucleotide specific for a twentieth mRNA, the twentieth mRNA being selected from the mRNAs in the RNA signature. In some embodiments, the plurality further comprises a twenty-first polynucleotide specific for a twenty-first mRNA, the twenty-first mRNA being selected from the mRNAs in the RNA signature. In some embodiments, the plurality further comprises a twenty-second polynucleotide specific for a twenty-second mRNA, the twenty-second mRNA being selected from the mRNAs in the RNA signature. In some embodiments, the plurality further comprises a twenty-third polynucleotide specific for a twenty-third mRNA, the twenty-third mRNA being selected from the mRNAs in the RNA signature. In some embodiments, the plurality further comprises a twenty-fourth polynucleotide specific for a twenty-fourth mRNA, the twenty-fourth mRNA being selected from the mRNAs in the RNA signature. In some embodiments, the plurality further comprises a twenty-fifth polynucleotide specific for a twenty-fifth mRNA, the twenty-fifth mRNA being selected from the mRNAs in the RNA signature. In some embodiments, the plurality further comprises a twenty-sixth polynucleotide specific for a twenty-sixth mRNA, the twenty-sixth mRNA being selected from the mRNAs in the RNA signature. In some embodiments, the plurality further comprises a twenty-seventh polynucleotide specific for a twenty-seventh mRNA, the twenty-seventh mRNA being selected from the mRNAs in the RNA signature. In some embodiments, the plurality further comprises a twenty-eighth polynucleotide specific for a twenty-eighth mRNA, the twenty-eighth mRNA being selected from the mRNAs in the RNA signature. In some embodiments, the plurality further comprises a twenty-ninth polynucleotide specific for a twenty-ninth mRNA, the twenty-ninth mRNA being selected from the mRNAs in the RNA signature. In some embodiments, the plurality further comprises a thirtieth polynucleotide specific for a thirtieth mRNA, the thirtieth mRNA being selected from the mRNAs in the RNA signature. In some embodiments, the plurality further comprises a thirty-first polynucleotide specific for a thirty-first mRNA, the thirty-first mRNA being selected from the mRNAs in the RNA signature. In some embodiments, the plurality further comprises a thirty-second polynucleotide specific for a thirty-second mRNA, the thirty-second mRNA being selected from the mRNAs in the RNA signature. In some embodiments, the plurality further comprises a thirty-third polynucleotide specific for a thirty-third mRNA, the thirty-third mRNA being selected from the mRNAs in the RNA signature. In some embodiments, the plurality further comprises a thirty-fourth polynucleotide specific for a thirty-fourth mRNA, the thirty-fourth mRNA being selected from the mRNAs in the RNA signature. In some embodiments, the plurality further comprises a thirty-fifth polynucleotide specific for a thirty-fifth mRNA, the thirty-fifth mRNA being selected from the mRNAs in the RNA signature. In some embodiments, the plurality further comprises a thirty-sixth polynucleotide specific for a thirty-sixth mRNA, the thirty-sixth mRNA being selected from the mRNAs in the RNA signature. In some embodiments, the plurality further comprises a thirty-seventh polynucleotide specific for a thirty-seventh mRNA, the thirty-seventh mRNA being selected from the mRNAs in the RNA signature. In some embodiments, the plurality further comprises a thirty-eighth polynucleotide specific for a thirty-eighth mRNA, the thirty-eighth mRNA being selected from the mRNAs in the RNA signature. In some embodiments, the plurality further comprises a thirty-ninth polynucleotide specific for a thirty-ninth mRNA, the thirty-ninth mRNA being selected from the mRNAs in the RNA signature. In some embodiments, the plurality further comprises a fortieth polynucleotide specific for a fortieth mRNA, the fortieth mRNA being selected from the mRNAs in the RNA signature. In some embodiments, the plurality further comprises a forty-first polynucleotide specific for a forty-first mRNA, the forty-first mRNA being selected from the mRNAs in the RNA signature. In some embodiments, the plurality further comprises a forty-second polynucleotide specific for a forty-second mRNA, the forty-second mRNA being selected from the mRNAs in the RNA signature. In some embodiments, the plurality further comprises a forty-third polynucleotide specific for a forty-third mRNA, the forty-third mRNA being selected from the mRNAs in the RNA signature. In some embodiments, the plurality further comprises a forty-fourth polynucleotide specific for a forty-fourth mRNA, the forty-fourth mRNA being selected from the mRNAs in the RNA signature.

It is understood that the use of ordinals (“first,” “second,” etc.) to designate polynucleotides or mRNAs indicates that the polynucleotides or mRNAs, as the case may be, are not identical to each other. It is also understood that in embodiments in which the “first,” “second,” etc. polynucleotides are primers (e.g., primers for carrying out an amplification reaction, such as PCR are related methods), the compositions can also optionally include one or more corresponding primers that hybridize to the opposite strand in order to facilitate amplification. Accordingly, the indication of a “first,” “second,” etc. polynucleotide may be considered as referring to a set of polynucleotides in such instances. It is further understood that in embodiments in which the “first,” “second,” etc. polynucleotides are capture probes, the compositions can also optionally include one or more corresponding detection probes that hybridize to same target in order to facilitate detection and quantitation.

In some embodiments, a composition includes cells or tissue obtained from a subject (e.g., a human patient). In some embodiments, a composition comprises mRNA isolated from a subject (e.g., a human patient). In some embodiments, a composition comprises cDNA synthesized from mRNA isolated from a subject (e.g., a human patient). In some embodiments, a composition includes control cells, tissues, mRNA, or cDNA.

In some embodiments, a composition comprises at least one polynucleotide or a plurality of polynucleotides suitable for use in detecting at least one reference mRNA. In some embodiments, a composition comprises reagents for performing hybridization and/or amplification, such as quantitative RT-PCR, microarray, digital PCR, RNA-Seq, RPA, Northern blot, or in situ hybridization ISH. Such reagents can include one or more of an enzyme with reverse transcriptase activity, a DNA polymerase (which may be thermophilic), an intercalating dye, dNTPs, buffer, a single-strand RNA nuclease, detergent, fixative (e.g., formaldehyde), cosolvent (e.g., formamide), etc.

In some embodiments, a kit is provided including one or more containers comprising at least one polynucleotide specific for an mRNA selected from the mRNAs in the RNA signature or a plurality of polynucleotides, the plurality comprising at least a first polynucleotide specific for a first mRNA and a second polynucleotide specific for a second mRNA, the first and second mRNAs being selected from the mRNAs in the RNA signature. In some embodiments, the plurality further comprises a third polynucleotide specific for a third mRNA, the third mRNA being selected from the mRNAs in the RNA signature. In some embodiments, the plurality further comprises a fourth polynucleotide specific for a fourth mRNA, the fourth mRNA being selected from the mRNAs in the RNA signature. In some embodiments, the plurality further comprises a fifth polynucleotide specific for a fifth mRNA, the fifth mRNA being selected from the mRNAs in the RNA signature. In some embodiments, the plurality further comprises a sixth polynucleotide specific for a sixth mRNA, the sixth mRNA being selected from the mRNAs in the RNA signature. In some embodiments, the plurality further comprises a seventh polynucleotide specific for a seventh mRNA, the seventh mRNA being selected from the mRNAs in the RNA signature. In some embodiments, the plurality further comprises an eighth polynucleotide specific for an eighth mRNA, the eighth mRNA being selected from the mRNAs in the RNA signature. In some embodiments, the plurality further comprises a ninth polynucleotide specific for a ninth mRNA, the ninth mRNA being selected from the mRNAs in the RNA signature. In some embodiments, the plurality further comprises a tenth polynucleotide specific for a tenth mRNA, the tenth mRNA being selected from the mRNAs in the RNA signature. In some embodiments, the plurality further comprises an eleventh polynucleotide specific for an eleventh mRNA, the eleventh mRNA being selected from the mRNAs in the RNA signature. In some embodiments, the plurality further comprises a twelfth polynucleotide specific for a twelfth mRNA, the twelfth mRNA being selected from the mRNAs in the RNA signature. In some embodiments, the plurality further comprises a thirteenth polynucleotide specific for a thirteenth mRNA, the thirteenth mRNA being selected from the mRNAs in the RNA signature. In some embodiments, the plurality further comprises a fourteenth polynucleotide specific for a fourteenth mRNA, the fourteenth mRNA being selected from the mRNAs in the RNA signature. In some embodiments, the plurality further comprises a fifteenth polynucleotide specific for a fifteenth mRNA, the fifteenth mRNA being selected from the mRNAs in the RNA signature. In some embodiments, the plurality further comprises a sixteenth polynucleotide specific for a sixteenth mRNA, the sixteenth mRNA being selected from the mRNAs in the RNA signature. In some embodiments, the plurality further comprises a seventeenth polynucleotide specific for a seventeenth mRNA, the seventeenth mRNA being selected from the mRNAs in the RNA signature. In some embodiments, the plurality further comprises an eighteenth polynucleotide specific for an eighteenth mRNA, the eighteenth mRNA being selected from the mRNAs in the RNA signature. In some embodiments, the plurality further comprises a nineteenth polynucleotide specific for a nineteenth mRNA, the nineteenth mRNA being selected from the mRNAs in the RNA signature. In some embodiments, the plurality further comprises an eighteenth polynucleotide specific for an eighteenth mRNA, the eighteenth mRNA being selected from the mRNAs in the RNA signature. In some embodiments, the plurality further comprises a twentieth polynucleotide specific for a twentieth mRNA, the twentieth mRNA being selected from the mRNAs in the RNA signature. In some embodiments, the plurality further comprises a twenty-first polynucleotide specific for a twenty-first mRNA, the twenty-first mRNA being selected from the mRNAs in the RNA signature. In some embodiments, the plurality further comprises a twenty-second polynucleotide specific for a twenty-second mRNA, the twenty-second mRNA being selected from the mRNAs in the RNA signature. In some embodiments, the plurality further comprises a twenty-third polynucleotide specific for a twenty-third mRNA, the twenty-third mRNA being selected from the mRNAs in the RNA signature. In some embodiments, the plurality further comprises a twenty-fourth polynucleotide specific for a twenty-fourth mRNA, the twenty-fourth mRNA being selected from the mRNAs in the RNA signature. In some embodiments, the plurality further comprises a twenty-fifth polynucleotide specific for a twenty-fifth mRNA, the twenty-fifth mRNA being selected from the mRNAs in the RNA signature. In some embodiments, the plurality further comprises a twenty-sixth polynucleotide specific for a twenty-sixth mRNA, the twenty-sixth mRNA being selected from the mRNAs in the RNA signature. In some embodiments, the plurality further comprises a twenty-seventh polynucleotide specific for a twenty-seventh mRNA, the twenty-seventh mRNA being selected from the mRNAs in the RNA signature. In some embodiments, the plurality further comprises a twenty-eighth polynucleotide specific for a twenty-eighth mRNA, the twenty-eighth mRNA being selected from the mRNAs in the RNA signature. In some embodiments, the plurality further comprises a twenty-ninth polynucleotide specific for a twenty-ninth mRNA, the twenty-ninth mRNA being selected from the mRNAs in the RNA signature. In some embodiments, the plurality further comprises a thirtieth polynucleotide specific for a thirtieth mRNA, the thirtieth mRNA being selected from the mRNAs in the RNA signature. In some embodiments, the plurality further comprises a thirty-first polynucleotide specific for a thirty-first mRNA, the thirty-first mRNA being selected from the mRNAs in the RNA signature. In some embodiments, the plurality further comprises a thirty-second polynucleotide specific for a thirty-second mRNA, the thirty-second mRNA being selected from the mRNAs in the RNA signature. In some embodiments, the plurality further comprises a thirty-third polynucleotide specific for a thirty-third mRNA, the thirty-third mRNA being selected from the mRNAs in the RNA signature. In some embodiments, the plurality further comprises a thirty-fourth polynucleotide specific for a thirty-fourth mRNA, the thirty-fourth mRNA being selected from the mRNAs in the RNA signature. In some embodiments, the plurality further comprises a thirty-fifth polynucleotide specific for a thirty-fifth mRNA, the thirty-fifth mRNA being selected from the mRNAs in the RNA signature. In some embodiments, the plurality further comprises a thirty-sixth polynucleotide specific for a thirty-sixth mRNA, the thirty-sixth mRNA being selected from the mRNAs in the RNA signature. In some embodiments, the plurality further comprises a thirty-seventh polynucleotide specific for a thirty-seventh mRNA, the thirty-seventh mRNA being selected from the mRNAs in the RNA signature. In some embodiments, the plurality further comprises a thirty-eighth polynucleotide specific for a thirty-eighth mRNA, the thirty-eighth mRNA being selected from the mRNAs in the RNA signature. In some embodiments, the plurality further comprises a thirty-ninth polynucleotide specific for a thirty-ninth mRNA, the thirty-ninth mRNA being selected from the mRNAs in the RNA signature. In some embodiments, the plurality further comprises a fortieth polynucleotide specific for a fortieth mRNA, the fortieth mRNA being selected from the mRNAs in the RNA signature. In some embodiments, the plurality further comprises a forty-first polynucleotide specific for a forty-first mRNA, the forty-first mRNA being selected from the mRNAs in the RNA signature. In some embodiments, the plurality further comprises a forty-second polynucleotide specific for a forty-second mRNA, the forty-second mRNA being selected from the mRNAs in the RNA signature. In some embodiments, the plurality further comprises a forty-third polynucleotide specific for a forty-third mRNA, the forty-third mRNA being selected from the mRNAs in the RNA signature. In some embodiments, the plurality further comprises a forty-fourth polynucleotide specific for a forty-fourth mRNA, the forty-fourth mRNA being selected from the mRNAs in the RNA signature.

It is understood that the use of ordinals (“first,” “second,” etc.) to designate polynucleotides or mRNAs indicates that the polynucleotides or mRNAs, as the case may be, are not identical to each other. It is also understood that in embodiments in which the “first,” “second,” etc. polynucleotides are primers (e.g., primers for carrying out an amplification reaction, such as PCR are related methods), the kits can also optionally include one or more corresponding primers that hybridize to the opposite strand in order to facilitate amplification. Accordingly, the indication of a “first,” “second,” etc. polynucleotide may be considered as referring to a set of polynucleotides in such instances. It is further understood that in embodiments in which the “first,” “second,” etc. polynucleotides are capture probes, the kits can also optionally include one or more corresponding detection probes that hybridize to same target in order to facilitate detection and quantitation.

In some embodiments, the kit includes one or more containers comprising at least one polynucleotide or a plurality of polynucleotides suitable for use in detecting at least one reference mRNA. In some embodiments, the kit comprises one or more containers comprising reagents for performing hybridization and/or amplification, such as quantitative RT-PCR, microarray, digital PCR, rNA-Seq, RNase Protection Assay (RPA), Northern blot, and in situ hybridization (ISH). Such reagents can include one or more of an enzyme with reverse transcriptase activity, a DNA polymerase (which may be thermophilic), an intercalating dye, dNTPs, buffer, a single-strand RNA nuclease, detergent, fixative (e.g., formaldehyde), co-solvent (e.g., formamide), etc. The kits of the invention can optionally include control samples to which the RNA signature score of a test sample can be compared in order to determine whether the RNA signature score of the test sample is elevated.

In some embodiments, the kits include compositions for detecting protein levels. Accordingly, the kits may include, e.g., antibodies specific for the components of an expression signature as described herein, in any of the numbers listed above in reference to the RNA signatures.

In addition to components for use in detection of RNA signature components (or corresponding proteins), the kits of the invention can also optionally include one or more therapeutic agents for administration to a subject if, e.g., a sample of the subject is found to have an elevated signature score. These therapeutic agents can include one or more LILRB2 antibody and/or one or more PD-1 antagonists, such as those described herein. These components can optionally be present in the kits in dosage form to facilitate administration. For example, in some embodiments, such a unit dosage is supplied in single-use prefilled syringe for injection. In some embodiments, the composition contained in the unit dosage can include saline, sucrose, or the like; a buffer, such as phosphate, or the like; and/or be formulated within a stable and effective pH range. In some embodiments, the composition can be provided as a lyophilized powder that may be reconstituted upon addition of an appropriate liquid, for example, sterile water. In some embodiments, the composition includes one or more substances that inhibit protein aggregation, including, for example, sucrose and arginine. In some embodiments, a composition includes heparin and/or a proteoglycan. In some embodiments, the amount of the LILRB2 antibody and/or PD-1 antagonist used in the unit dose can be any of the amounts provided herein for the various methods and/or compositions described.

In some embodiments, kits further include instructions for use in the determination of RNA signature score and, optionally, the treatment of cancer with a LILRB2 antibody and PD-1 antagonist therapy. The kits may further include a description of selection a subject suitable for treatment. Instructions supplied in the kits are typically written instructions on a label or package insert (for example, a paper sheet included in the kit), but machine-readable instructions (for example, instructions carried on a magnetic or optical storage disk) are also acceptable. The kits are in suitable packaging, which may include, for example, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### III. METHODS OF TREATMENT

Subjects having elevated LILRB2 relative to IFNg are treated with a LILRB2 antibody and a PD1 antagonist, while subjects having a balanced level of LILRB2 and IFNg, or elevated IFNg relative to LILRB2, are treated with a PD1 antagonist, in the absence of a LILRB2 antibody, as described herein. Elevated LILRB2 can be indicated directly, by LILRB2 levels per se compared to IFNg levels, or indirectly, by high TAM or immunosuppressive myeloid levels compared to IFNg levels. This treatment can be used in methods for preventing, improving, or treating cancer in the subjects. Preferably the subjects treated by the methods described herein are human patients.

Subjects that can be treated as described herein thus include patients having cancer. The type of cancer can be any type of cancer listed herein or otherwise known in the art. Exemplary types of cancer include, but are not limited to, gastric cancer, melanoma (e.g., skin cutaneous melanoma), urothelial cancer, lymphoid neoplasm, diffuse large B-cell lymphoma (DLBCL), testicular germ cell tumors (TGCT), mesothelioma, kidney cancer (e.g., kidney renal clear cell carcinoma, kidney renal papillary cell carcinoma, or renal cell carcinoma (RCC)), sarcoma, lung cancer (e.g., lung adenocarcinoma, lung squamous cell carcinoma, and non-small cell lung cancer (NSCLC)) stomach adenocarcinoma, pancreatic adenocarcinoma, head and neck squamous cell carcinoma, ovarian serious cystadenocarcinoma, liver hepatocellular carcinoma, skin cutaneous melanoma, colon adenocarcinoma, breast cancer (e.g., breast invasive carcinoma or triple negative breast cancer), rectum adenocarcinoma, glioblastoma multiforme, uterine corpus endometrial carcinoma, thymoma, bladder cancer, endometrial cancer, Hodgkin's lymphoma, ovarian cancer, anal cancer, biliary cancer, colorectal cancer, and esophageal cancer. Also see the definition of cancer, above, for additional cancer types that can be treated according to the methods of the invention.

Patients that can be treated as described herein include patients who have not previously received a different anti-cancer therapy and patients who have received previous (e.g., 1, 2, 3, 4, 5, or more) doses or cycles of one or more (e.g., 1, 2, 3, 4, 5, or more) of different anti-cancer therapies including, e.g., treatment with one or more LILRB2 antibody and/or PD-1 antagonist (or any other agent described herein).

The combination treatments of the methods described herein can be concurrent or sequential, as determined to be appropriate by those of skill in the art. Thus, in some embodiments, one or more LILRB2 antibody (e.g., as described herein) can be administered at the same time as, before, or after one or more PD-1 antagonist (e.g., as described herein). If administered before or after, the administration of the LILRB2 antibody and PD-1 targeted agents can overlap in time at least in part, and thus be concurrent. Alternatively, the administrations can be such that they do not overlap, and thus be sequential. In other embodiments, one or more PD-1 antagonist (e.g., as described herein) can be administered before or after one or more LILRB2 antibody (e.g., as described herein), whether concurrently or sequentially.

In addition to treatment with LILRB2 and PD-1 antagonist therapy, or PD-1 antagonist therapy in the absence of LILRB2 antibody, any one or more of the anti-cancer therapies listed herein (see below) and others known in the art can be used in combination with the methods of the invention. In some embodiments, the one or more additional anti-cancer therapies is two or more anti-cancer therapies. In some embodiments, the one or more additional anti-cancer therapies is three or more anti-cancer therapies. Specific, non-limiting examples of additional anti-cancer therapies that can be used in the invention including, e.g., immunotherapies, chemotherapies, and cancer vaccines, among others, are provided below. In some embodiments, the one or more additional anti-cancer therapies is administered prior to the therapy of the invention. In some embodiments, the one or more additional anti-cancer therapies is administered at the same time as the therapy of the invention. In some embodiments, the one or more additional anti-cancer therapies is administered after the therapy of the invention.

In some embodiments, the therapy of the invention (and/or the one or more additional anti-cancer therapies) is administered to the patient multiple times at regular intervals. These multiple administrations can also be referred to as administration cycles or therapy cycles. In some embodiments, the therapy of the invention (and/or the one or more anti-cancer therapies) is administered to the patient for more than two cycles, more than three cycles, more than four cycles, more than five cycles, more than ten cycles, more than fifteen cycles, or more than twenty cycles.

In some embodiments, the regular interval is a dosage every week, a dosage every two weeks, a dosage every three weeks, a dosage every four weeks, a dosage every five weeks, a dosage every six weeks, a dosage every seven weeks, a dosage every eight weeks, a dosage every nine weeks, a dosage every ten weeks, a dosage every eleven weeks, or a dosage every twelve weeks.

### Therapeutic Anti-LILRB2 Antibodies

Therapeutic anti-LILRB2 antibodies that can be used in the invention include, but are not limited to, humanized antibodies, chimeric antibodies, human antibodies, and antibodies comprising any of the heavy chain and/or light chain CDRs discussed herein. In some embodiments, the antibody is an isolated antibody. In some embodiments, the antibody is a monoclonal antibody. In some embodiments, the antibody is selected from JTX-8064 (WO2019126514A2), LILRB2 mAB (WO2020014132A2), MK-4830 (Merck Sharp and Dohme), NGM707 (NGM Biopharmaceuticals/Merck), 10-108 (Immune-onc Therapeutics), and iosH2 (ImmuneOs). In some embodiments, the antibody is selected from an antibody described in one or more of the following documents, each of which is incorporated herein by reference: WO2019126514A2, WO2020014132A2, WO2018022881, WO2020061059A1, WO2013181438, WO2019144052A1, and WO2003000199A2. Preferably the anti-LILRB2 antibody specifically binds to human LILRB2 (e.g., accession number Q8N423 or NP_005865.3)

In some embodiments, the therapeutic anti-LILRB2 antibody comprises a variable heavy chain (V_{H}) domain sequence having at least 90%, 91%, 92%, 93%, 94%, 95%, 96%, 97%, 98%, 99%, or 100% sequence identity to the amino acid sequence of SEQ ID NO: 3. In some embodiments, a V_{H} sequence having at least 90%, 91%, 92%, 93%, 94%, 95%, 96%, 97%, 98%, or 99% identity contains substitutions (for example, conservative substitutions), insertions, or deletions relative to the reference sequence, but an anti-LILRB2 antibody comprising that sequence retains the ability to bind to LILRB2. In some embodiments, a total of 1 to 10 amino acids have been substituted, inserted, and/or deleted in SEQ ID NO: 3. In some embodiments, substitutions, insertions, or deletions occur in regions outside the CDRs (that is, in the FRs). Optionally, the anti-LILRB2 antibody comprises the V_{H} sequence of SEQ ID NO: 3, including post-translational modifications of that sequence.

In some embodiments, the V_{H} comprises: (a) CDR-H1 comprising the amino acid sequence of SEQ ID NO: 5; (b) CDR-H2 comprising the amino acid sequence of SEQ ID NO: 6; and (c) CDR-H3 comprising the amino acid sequence of SEQ ID NO: 7.

In some embodiments, an anti-LILRB2 antibody is provided, wherein the antibody comprises a variable light chain (V_{L}) domain having at least 90%, 91%, 92%, 93%, 94%, 95%, 96%, 97%, 98%, 99%, or 100% sequence identity to the amino acid sequence of SEQ ID NO: 4. In some embodiments, a V_{L} sequence having at least 90%, 91%, 92%, 93%, 94%, 95%, 96%, 97%, 98%, or 99% identity contains substitutions (for example, conservative substitutions), insertions, or deletions relative to the reference sequence, but an anti-LILRB2 antibody comprising that sequence retains the ability to bind to LILRB2. In some embodiments, a total of 1 to 10 amino acids have been substituted, inserted, and/or deleted in SEQ ID NO: 4. In some embodiments, the substitutions, insertions, or deletions occur in regions outside the CDRs (that is, in the FRs). Optionally, the anti-LILRB2 antibody comprises the V_{L} sequence of SEQ ID NO: 4, including post-translational modifications of that sequence.

In some embodiments, the V_{L} comprises: (a) CDR-L1 comprising the amino acid sequence of SEQ ID NO: 8; (b) CDR-L2 comprising the amino acid sequence of SEQ ID NO: 9; and (c) CDR-L3 comprising the amino acid sequence of SEQ ID NO: 10.

In some embodiments, an anti-LILRB2 antibody comprises a V_{H} domain sequence having at least 90%, 91%, 92%, 93%, 94%, 95%, 96%, 97%, 98%, 99%, or 100% sequence identity to the amino acid sequence of SEQ ID NO: 3 and a V_{L} having at least 90%, 91%, 92%, 93%, 94%, 95%, 96%, 97%, 98%, 99%, or 100% sequence identity to the amino acid sequence of SEQ ID NO: 4. In some embodiments, a V_{H} domain sequence having at least 90%, 91%, 92%, 93%, 94%, 95%, 96%, 97%, 98%, or 99% identity contains substitutions (for example, conservative substitutions), insertions, or deletions relative to the reference sequence, and a V_{L} domain sequence having at least 90%, 91%, 92%, 93%, 94%, 95%, 96%, 97%, 98%, or 99% identity contains substitutions (for example, conservative substitutions), insertions, or deletions relative to the reference sequence, but an anti-LILRB2 antibody comprising that sequence retains the ability to bind to LILRB2. In some embodiments, a total of 1 to 10 amino acids have been substituted, inserted, and/or deleted in SEQ ID NO: 3. In some embodiments, a total of 1 to 10 amino acids have been substituted, inserted, and/or deleted in SEQ ID NO: 4. In some embodiments, substitutions, insertions, or deletions occur in regions outside the CDRs (that is, in the FRs). Optionally, the anti-LILRB2 antibody comprises the V_{H} domain sequence in SEQ ID NO: 3 and the V_{L} domain sequence of SEQ ID NO: 4, including post-translational modifications of one or both sequences.

In some embodiments, an anti-LILRB2 antibody comprises a V_{H} domain as in any of the embodiments provided herein, and a V_{L} domain as in any of the embodiments provided herein. In some embodiments, the antibody comprises the V_{H} and V_{L} domain sequences of SEQ ID NO: 3 and SEQ ID NO: 4, respectively, including post-translational modifications of those sequences.

In some embodiments, an anti-LILRB2 antibody comprises a heavy chain comprising the amino acid sequence of SEQ ID NO: 1 and a light chain comprising the amino acid sequence of SEQ ID NO: 2. In each instance of reference to SEQ ID NO: 1 herein, the sequence of SEQ ID NO: 1 can optionally consist solely of SEQ ID NO: 1 as shown in the sequence listing below, or it can optionally also include a C-terminal lysine added to the listed sequence of SEQ ID NO: 1.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated. In general, anti-LILRB2 antibodies may be administered in an amount in the range of about 10 μg/kg body weight to about 100 mg/kg body weight per dose. In some embodiments, anti-LILRB2 antibodies may be administered in an amount in the range of about 50 μg/kg body weight to about 5 mg/kg body weight per dose. In some embodiments, anti-LILRB2 antibodies may be administered in an amount in the range of about 100 μg/kg body weight to about 10 mg/kg body weight per dose. In some embodiments, anti-LILRB2 antibodies may be administered in an amount in the range of about 100 μg/kg body weight to about 20 mg/kg body weight per dose. In some embodiments, anti-LILRB2 antibodies may be administered in an amount in the range of about 0.5 mg/kg body weight to about 20 mg/kg body weight per dose. Additional details concerning dosage are provided below.

In some embodiments, pharmaceutical compositions are administered in an amount effective to cause conversion of M2-like macrophages to M1-like macrophages.

The therapeutically effective amount is typically dependent on the weight of the subject being treated, his or her physical or health condition, the extensiveness of the condition to be treated, or the age of the subject being treated. In general, anti-LILRB2 antibodies may be administered in an amount in the range of about 10 μg/kg body weight to about 100 mg/kg body weight per dose. In some embodiments, anti-LILRB2 antibodies may be administered in an amount in the range of about 50 μg/kg body weight to about 5 mg/kg body weight per dose. In some embodiments, anti-LILRB2 antibodies may be administered in an amount in the range of about 100 μg/kg body weight to about 10 mg/kg body weight per dose. In some embodiments, anti-LILRB2 antibodies may be administered in an amount in the range of about 100 μg/kg body weight to about 20 mg/kg body weight per dose. In some embodiments, anti-LILRB2 antibodies may be administered in an amount in the range of about 0.5 mg/kg body weight to about 20 mg/kg body weight per dose.

In some embodiments, a LILRB2 antibody (e.g., JTX-8064) is administered alone or in combination with a PD-1 antagonist (or another anti-cancer agent, e.g., as described herein) in a unit dosage amount of 300 to 1200 mg, e.g., 400 to 900 mg or 450 to 900 mg. In some embodiments, the unit dosage of a LILRB2 antibody (e.g., JTX-8064) is within a range selected from the group consisting of 300-450 mg, 350-500 mg, 400-550 mg, 450-600 mg, 500-650 mg, 550-700 mg, 600-750 mg, 650-800 mg, 700-850 mg, 750-900 mg, 800-950 mg, 850-1000 mg, 900-1050 mg, 950-1100 mg, 1000-1150 mg, 1050-1150 mg, 1100-100 mg, and 950-1200 mg. In some embodiments, the unit dosage of a LILRB2 antibody (e.g., JTX-8064) is within a range selected from the group consisting of 300-400 mg, 350-450 mg, 400-500 mg, 450-550 mg, 500-600 mg, 550-650 mg, 600-700 mg, 650-750 mg, 700-800 mg, 750-850 mg, 800-900 mg, 850-950 mg, 900-1000 mg, 950-1050 mg, 1000-1100 mg, 1050-1150 mg, and 1100-1200 mg. In some embodiments, the unit dosage of a LILRB2 antibody (e.g., JTX-8064) is within the range of 600-800 mg. In some embodiments, the unit dosage of a LILRB2 antibody (e.g., JTX-8064) is within the range of 650-750 mg. In some embodiments, the unit dosage of a LILRB2 antibody (e.g., JTX-8064) is selected from the group consisting of 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, and 1200 mg. In some embodiments, the unit dosage of a LILRB2 antibody is 700 mg.

It is of note that target saturation with JTX-8064 was observed at doses of 300 mg and above (throughout a 21-day dosing interval) in human patient studies.

In some embodiments, a LILRB2 antibody (e.g., JTX-8064) is administered alone or in combination with a PD-1 antagonist (or another anti-cancer agent, e.g., as described herein) at a dose of 5-15 mg/kg. In some embodiments, the LILRB2 antibody (e.g., JTX-8064) is administered once every three weeks. In some embodiments, the LILRB2 antibody (e.g., JTX-8064) is administered alone or in combination with a PD-1 antagonist (or another anti-cancer agent, e.g., as described herein) at a dose within a range selected from the group consisting of: 5-10 mg/kg, 7.5-12.5 mg/kg, and 10-15 mg/kg. In some embodiments, the LILRB2 antibody (e.g., JTX-8064) is administered once every three weeks.

### PD-1 Therapies

A PD-1 therapy encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies. Preferably, the PD-1 therapy is directed to and/or specific for human PD-1 or PD-L1.

Non-limiting, exemplary PD-1 therapies include nivolumab (OPDIVO®, BMS-936558, MDX-1106, ONO-4538); pidilizumab, lambrolizumab/pembrolizumab (KEYTRUDA, MK-3475); BGB-A317, tislelizumab (BeiGene/Celgene); durvalumab (anti-PD-L1 antibody, MEDI-4736; AstraZeneca/MedImmune); RG-7446; avelumab (anti-PD-L1 antibody; MSB-0010718C; Pfizer); AMP-224; BMS-936559 (anti-PD-L1 antibody); AMP-514; MDX-1105; A B-011; anti-LAG-3/PD-1; spartalizumab (CoStim/Novartis); anti-PD-1 antibody (Kadmon Pharm.); anti-PD-1 antibody (Immunovo); anti-TEVI-3/PD-1 antibody (AnaptysBio); anti-PD-L1 antibody (CoStim/Novartis); RG7446/MPDL3280A (anti-PD-L1 antibody, Genentech/Roche); KD-033 (Kadmon Pharm.); AGEN-2034 (Agenus); STI-A1010; STI-A1110; TSR-042; atezolizumab (TECENTRIQ™); and other antibodies that are directed against programmed death-1 (PD-1) or programmed death ligand 1 (PD-L1).

PD-1 therapies are administered according to regimens that are known in the art, e.g., US FDA-approved regimens. In one example, nivolumab is administered as an intravenous infusion over 60 minutes in the amount of 240 mg every two weeks (unresectable or metastatic melanoma, adjuvant treatment for melanoma, non-small cell lung cancer (NSCLC), advanced renal cell carcinoma, locally advanced renal cell carcinoma, MSI-H or dMMR metastatic colorectal cancer, and hepatocellular carcinoma) or in the amount of 3 mg/kg every three weeks (classical Hodgkin lymphoma; recurrent or metastatic squamous cell carcinoma of the head and neck). In another example, pembrolizumab is administered by intravenous infusion over 30 minutes in the amount of 200 mg, once every three weeks. In another example, atezolizumab is administered by intravenous infusion over 60 minutes in the amount of 1200 mg every three weeks. In another example, avelumab is administered by intravenous infusion over 60 minutes in the amount of 10 mg/kg every two weeks. In another example, durvalumab is administered by intravenous infusion over 60 minutes in the amount of 10 mg/kg every two weeks.

### IV. EXEMPLARY ANTI-CANCER THERAPIES FOR USE IN COMBINATIONS

As examples, any anti-cancer therapy listed herein or otherwise known in the art, can be used in combination with a LILRB2 antibody and a PD-1 antagonist, or a PD-1 antagonist in the absence of a LILRB2 antibody, as described herein or as a pre-treatment or post-treatment. Examples of such additional therapeutic agents are provided below.

In various examples, the components of a combination are administered according to dosing regimens described herein (e.g., US FDA-approved dosing regimens; see above), or using other regimens determined to be appropriate by those of skill in the art. Exemplary anti-cancer therapies are described below.

a. Immunotherapies

In some embodiments, the one or more anti-cancer therapies is an immunotherapy. The interaction between cancer and the immune system is complex and multifaceted. See de Visser et al., Nat. Rev. Cancer 6:24-37, 2006. While many cancer patients appear to develop an anti-tumor immune response, cancers also develop strategies to evade immune detection and destruction. Recently, immunotherapy has been developed for the treatment and prevention of cancer and other disorders. Immunotherapy provides the advantage of cell specificity that other treatment modalities lack. As such, methods for enhancing the efficacy of immune based therapies can be clinically beneficial.

i. Anti-CTLA-4 Antagonist Antibodies

In some embodiments, the one or more anti-cancer therapies is an anti-CTLA-4 antagonist antibody. An anti-CTLA-4 antagonist antibody refers to an agent capable of inhibiting the activity of cytotoxic T-lymphocyte-associated protein 4 (CTLA-4), thereby activating the immune system. The CTLA-4 antagonist may bind to CTLA-4 and reverse CTLA-4-mediated immunosuppression. A non-limiting exemplary anti-CTLA-4 antibody is ipilimumab (YERVOY®, BMS), which may be administered according to methods known in the art, e.g., as approved by the US FDA. For example, ipilimumab may be administered in the amount of 3 mg/kg intravenously over 90 minutes every three weeks for a total of 4 doses (unresectable or metastatic melanoma); or at 10 mg/kg intravenously over 90 minutes every three weeks for a total of 4 doses, followed by 10 mg/kg every 12 weeks for up to 3 years or until documented recurrence or unacceptable toxicity (adjuvant melanoma).

ii. OX40 Agonist Antibodies

In some embodiments, the one or more anti-cancer therapies is an agonist anti-OX40 antibody. An OX40 agonist antibody refers to an agent that induces the activity of OX40, thereby activating the immune system and enhancing anti-tumor activity. Non-limiting, exemplary agonist anti-OX40 antibodies are Medi6469, MedImmune, and MOXR0916/RG7888, Roche. These antibodies may be administered according to methods and in regimens determined to be appropriate by those of skill in the art.

iii. TIGIT Antagonists

In some embodiments, the one or more anti-cancer therapies is TIGIT antagonist. A TIGIT antagonist refers to an agent capable of antagonizing or inhibiting the activity of T-cell immunoreceptor with Ig and ITIM domains (TIGIT), thereby reversing TIGIT-mediated immunosuppression. A non-limiting exemplary TIGIT antagonist is BMS-986207 (Bristol-Myers Squibb/Ono Pharmaceuticals). These agents may be administered according to methods and in regimens determined to be appropriate by those of skill in the art.

iv. IDO inhibitors

In some embodiments, the one or more anti-cancer therapies is an IDO inhibitor. An IDO inhibitor refers to an agent capable of inhibiting the activity of indoleamine 2,3-dioxygenase (IDO) and thereby reversing IDO-mediated immunosuppression. The IDO inhibitor may inhibit IDO1 and/or ID02 (INDOL1). An IDO inhibitor may be a reversible or irreversible IDO inhibitor. A reversible IDO inhibitor is a compound that reversibly inhibits IDO enzyme activity either at the catalytic site or at a non-catalytic site while an irreversible IDO inhibitor is a compound that irreversibly inhibits IDO enzyme activity by forming a covalent bond with the enzyme. Non-limiting exemplary IDO inhibitors are described, e.g., in US 2016/0060237; and US 2015/0352206. Non-limiting exemplary IDO inhibitors include Indoximod (New Link Genetics), INCB024360 (Incyte Corp), 1-methyl-D-tryptophan (New Link Genetics), and GDC-0919/navoximod (Genentech/New Link Genetics). These agents may be administered according to methods and in regimens determined to be appropriate by those of skill in the art.

v. RORγ Agonists

In some embodiments, the one or more anti-cancer therapies is a RORγ agonist. RORγ agonists refer to an agent capable of inducing the activity of retinoic acid-related orphan receptor gamma (RORγ), thereby decreasing immunosuppressive mechanisms. Non-limiting exemplary RORγ agonists include, but are not limited to, LYC-55716 (Lycera/Celgene) and INV-71 (Innovimmune). These agents may be administered according to methods and in regimens determined to be appropriate by those of skill in the art.

b. Chemotherapies

In some embodiments, the one or more anti-cancer therapies is a chemotherapeutic agent. Exemplary chemotherapeutic agents that can be used include, but are not limited to, capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXA E® (protein-bound paclitaxel), pemetrexed, vinorelbine, vincristine, erlotinib, afatinib, gefitinib, crizotinib, dabrafenib, trametinib, vemurafenib, and cobimetanib. These agents may be administered according to methods and in regimens determined to be appropriate by those of skill in the art.

c. Cancer Vaccines

In some embodiments, the one or more anti-cancer therapies is a cancer vaccine. Cancer vaccines have been investigated as a potential approach for antigen transfer and activation of dendritic cells. In particular, vaccination in combination with immunologic checkpoints or agonists for co-stimulatory pathways have shown evidence of overcoming tolerance and generating increased anti-tumor response. A range of cancer vaccines have been tested that employ different approaches to promoting an immune response against the tumor (see, e.g., Emens L A, Expert Opin Emerg Drugs 13(2): 295-308 (2008)). Approaches have been designed to enhance the response of B cells, T cells, or professional antigen-presenting cells against tumors. Exemplary types of cancer vaccines include, but are not limited to, peptide-based vaccines that employ targeting distinct tumor antigens, which may be delivered as peptides/proteins or as genetically-engineered DNA vectors, viruses, bacteria, or the like; and cell biology approaches, for example, for cancer vaccine development against less well-defined targets, including, but not limited to, vaccines developed from patient-derived dendritic cells, autologous tumor cells or tumor cell lysates, allogeneic tumor cells, and the like.

Exemplary cancer vaccines include, but are not limited to, dendritic cell vaccines, oncolytic viruses, tumor cell vaccines, etc. In some embodiments, such vaccines augment the anti-tumor response. Examples of cancer vaccines also include, but are not limited to, MAGE3 vaccine (e.g., for melanoma and bladder cancer), MUC1 vaccine (e.g., for breast cancer), EGFRv3 (such as Rindopepimut, e.g., for brain cancer, including glioblastoma multiforme), or ALVAC-CEA (e.g., for CEA+ cancers).

Non-limiting exemplary cancer vaccines also include Sipuleucel-T, which is derived from autologous peripheral-blood mononuclear cells (PBMCs) that include antigen-presenting cells (see, e.g., Kantoff P W et al., N Engl J Med. 363:411-22 (2010)). In Sipuleucel-T generation, the patients PBMCs are activated ex vivo with PA2024, a recombinant fusion protein of prostatic acid phosphatase (a prostate antigen) and granulocyte-macrophage colony-stimulating factor (an immune-cell activator). Another approach to a candidate cancer vaccine is to generate an immune response against specific peptides mutated in tumor tissue, such as melanoma (see, e.g., Carreno et al., Science 348:6236, 2015). Such mutated peptides may, in some embodiments, be referred to as neoantigens. As a non-limiting example of the use of neoantigens in tumor vaccines, neoantigens in the tumor predicted to bind the major histocompatibility complex protein HLA-A*02:01 are identified for individual patients with a cancer, such as melanoma. Dendritic cells from the patient are matured ex vivo, then incubated with neoantigens. The activated dendritic cells are then administered to the patient. In some embodiments, following administration of the cancer vaccine, robust T-cell immunity against the neoantigen is detectable.

In some such embodiments, the cancer vaccine is developed using a neoantigen. In some embodiments, the cancer vaccine is a DNA vaccine. In some embodiments, the cancer vaccine is an engineered virus comprising a cancer antigen, such as PROSTVAC (rilimogene galvacirepvec/rilimogene glafolivec). In some embodiments, the cancer vaccine comprises engineered tumor cells, such as GVAX, which is a granulocyte-macrophage colony-stimulating factor (GM-CSF) gene-transfected tumor cell vaccine (see, e.g., Nemunaitis, Expert Rev. Vaccines 4:259-274, 2005).

The vaccines may be administered according to methods and in regimens determined to be appropriate by those of skill in the art.

d. Additional Exemplary Anti-Cancer Therapies

Further non-limiting, exemplary anti-cancer therapies include Luspatercept (Acceleron Pharma/Celgene); Motolimod (Array BioPharma/CelgeneNentiRx Pharmaceuticals/Ligand); GI-6301 (Globelmmune/Celgene/NantWorks); GI-6200 (Globelmmune/Celgene/NantWorks); BLZ-945 (Celgene/Novartis); ARRY-382 (Array BioPharma/Celgene), or any of the anti-cancer therapies provided in Table 8. These agents may be administered according to methods and in regimens determined to be appropriate by those of skill in the art. In some embodiments, the one or more anti-cancer therapies includes surgery and/or radiation therapy. Accordingly, the anti-cancer therapies can optionally be utilized in the adjuvant or neoadjuvant setting.

### V. PHARMACEUTICAL COMPOSITIONS AND DOSING

Compositions including a LILRB2 antibody, a PD-1 antagonist, or a combination thereof (or one or more additional anti-cancer therapies as described herein) are provided in formulations with a wide variety of pharmaceutically acceptable carriers, as determined to be appropriate by those of skill in the art (see, for example, Gennaro, Remington: The Science and Practice of Pharmacy with Facts and Comparisons: Drugfacts Plus, 20^{th} ed. (2003); Ansel et al., Pharmaceutical Dosage Forms and Drug Delivery Systems, 7^{th} ed., Lippincott, Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

Anti-cancer therapies are administered in the practice of the methods of the present invention as is known in the art (e.g., according to FDA-approved regimens) or as indicated elsewhere herein (see, e.g., above). In some embodiments, anti-cancer therapies of the invention are administered in amounts effective for treatment of cancer. The therapeutically effective amount is typically dependent on the weight of the subject being treated, his or her physical or health condition, the extensiveness of the condition to be treated, the age of the subject being treated, pharmaceutical formulation methods, and/or administration methods (e.g., administration time and administration route).

In some embodiments, a composition of the invention comprises a LILRB2 antibody (e.g., JTX-8064) in a unit dosage amount of 300 to 1200 mg, e.g., 400 to 900 mg or 450 to 900 mg. In some embodiments, the unit dosage of a LILRB2 antibody (e.g., JTX-8064) is within a range selected from the group consisting of 300-450 mg, 350-500 mg, 400-550 mg, 450-600 mg, 500-650 mg, 550-700 mg, 600-750 mg, 650-800 mg, 700-850 mg, 750-900 mg, 800-950 mg, 850-1000 mg, 900-1050 mg, and 950-1200 mg. In some embodiments, the unit dosage of a LILRB2 antibody (e.g., JTX-8064) is within a range selected from the group consisting of 300-400 mg, 350-450 mg, 400-500 mg, 450-550 mg, 500-600 mg, 550-650 mg, 600-700 mg, 650-750 mg, 700-800 mg, 750-850 mg, 800-900 mg, 850-950 mg, 900-1000 mg, 950-1050 mg, 1000-1100 mg, 1050-1150 mg, and 1100-1200 mg. In some embodiments, the unit dosage of a LILRB2 antibody (e.g., JTX-8064) is within the range of 600-800 mg. In some embodiments, the unit dosage of a LILRB2 antibody (e.g., JTX-8064) is within the range of 650-750 mg. In some embodiments, the unit dosage of a LILRB2 antibody (e.g., JTX-8064) is selected from the group consisting of 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, and 1200 mg. In some embodiments, the unit dosage of a LILRB2 antibody is 700 mg.

In some embodiments, anti-cancer therapies can be administered in vivo by various routes, including, but not limited to, intravenous, intra-arterial, parenteral, intratumoral, intraperitoneal, or subcutaneous. The appropriate formulation and route of administration can be selected by those of skill in the art according to the intended application.

### VI. EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Methods

Data were used from studies with pre-treatment tumor biopsy samples analyzed by RNA sequencing. Patients in these studies were then treated with anti-PD-1 or anti-PD-L1 therapies and response was reported. Gene expression profiles were used to make correlations to outcomes on anti-PD(L)1 therapies to identify predictive biomarkers to immune checkpoint blockade.

Gene expression data (whole transcriptome RNA sequencing) were downloaded from published research articles and the public functional genomics data repository Gene Expression Omnibus (https://www.ncbi.nlm.nih.gov/geo/). LILRB2 was used as the sole member of the LILRB2 gene signature. IFNG signature was used from public research article relating to gene signatures connected to response to anti-PD-1 therapy (Ayers et al., J. Clin. Invest. 127(8):2930-2940, 2017).

The TAM signature includes a list of genes that are co-expressed with LILRB2 across human cancer single-cell RNAseq datasets. The list of single-cell RNAseq datasets used in this signature generation is described in Table 4. An internal pipeline based on the R package, Seurat, was used to filter low quality cells and normalize the filtered single-cell RNAseq expression datasets. Counts were normalized using the LogNormalize function in Seurat and scaled prior to calculating pairwise correlations between genes. Gene-wise scaling centers the distribution of expression of each gene to mean of 0 and variance of 1. The Spearman's correlation coefficients were calculated between the scaled expression of each gene and LILRB2 for each dataset. The top 50 genes with the highest mean of Spearman's correlation coefficients across datasets were retained for consideration as part of the TAM signature. Additional filtering steps were applied to the gene list including the exclusion genes with low expression in The Cancer Genome Atlas (TCGA) primary tumors (mean of log 2(FPKM+0.01)<0), exclusion of genes with low Spearman's correlation coefficients with LILRB2 expression from TCGA primary tumors (Spearman's correlation coefficient <0.5), and exclusion of genes not enriched in myeloid cells in selected single cell RNAseq datasets from Table 4. The enrichment of genes in myeloid cells is determined by a differential expression test between the normalized expression of the gene in myeloid cells vs. all other cells in each dataset. The final list of genes included in the TAM signature is defined in Table 2.

Gene expression data were analyzed in TPM (transcripts per million). Signatures were calculated per sample by gathering the gene expression values for all genes in the signature and taking the geometric mean of these values plus 0.0001:
Signature=geometric mean (x+0.0001)
X is equal to the values of all genes comprised in the gene signature
The 0.0001 is an additional, arbitrary factor that allows for the calculation of signature scores in the event that one of the genes has zero TPM.

Values of individual genes, in this instance LILRB2, are TPM and graphed in log 2(TPM). Ratio of LILRB2/IFNG signature was taken by dividing LILRB2 by IFNG signature and converting the ratio to log 2 scale:Log 2(LILRB2/IFNG Signature). Ratio of TAM signature/IFNG signature was taken by dividing TAM signature by IFNG signature and converting the ratio to log 2 scale:Log 2(TAM Signature/IFNG Signature). In clinical metadata associated with the datasets, responders were considered individuals with reported complete response (CR) or partial response (PR), and non-responders were considered individuals with reported stable disease (SD) or progressive disease (PD). Ratios were examined across response (responders vs non responders) by students t test.

### Results and Conclusions

It was observed that non-responders have significantly higher LILRB2 to IFNG signature ratios than responders (p<0.05 in 3/3 studies). Patients with a lower LILRB2 to IFNG signature ratio are more likely to have complete or partial responses to anti-PD(L)1 therapies. It was also observed that non-responders have significantly higher TAM signature to IFNG signature ratios than responders (p<0.05 in 2/3 studies). Patients with a lower TAM signature to IFNG signature ratio are more likely to have complete or partial responses to anti-PD(L)1 therapies.

The balance between LILRB2 or TAMs and IFNG, as observed by LILRB2/IFNG or TAM/IFNG ratios, can be used as a predictive biomarker of innate resistance to anti-PD(L)1 therapies. Immunosuppressive TAMs impede T effector activity and production of pro-inflammatory cytokines like IFNG and antagonism of LILRB2 may be able to correct these immunosuppressive impacts and bridge between innate and adaptive immune systems. JTX-8064, a LILRB2 antibody, can be used to shift the imbalance of this ratio by (i) reprogramming TAMs by functional blocking of LILRB2, and (ii) boosting IFNG production by T cells. Shifting this balance can improve patient responses in combination with PD-1 inhibitor.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

### REFERENCES

IFNG gene signature: Ayers et al., J. Clin. Invest. 127(8):2930-2940, 2017
Data used:
(1) Atezo Bladder, IMVIGOR
Reference: https://www.ncbi.nlm.nih.gov/pmc/articles/PMC6028240/; Mariathasan et al., Nature 544(7693):544-548, 2018
Data processing cited in: https://www.ncbi.nlm.nih.gov/pubmed/30851984; Kim et al., Eur. Urol. 75(6):961-964, 2019
(2) Gastric, Kim et al
Reference: Kim et al., Nat. Med. 24.9:1449-1458, 2018
Data: https://www.ebi.ac.uk/ena/browser/view/PRJEB25780
(3) Melanoma, Riaz
Reference: Riaz et al., Cell 171(4):934-949, 2017
Data: https://www.ncbi.nlm.nih.gov/geo/query/acc.cgi?acc=GSE91061

TABLE 2
TAM Signature

| | | | | | |
|---|---|---|---|---|---|
| CSF1R | IGSF6 | MS4A7 | CLEC7A | SLC7A7 | TLR2 |
| AIF1 | FCGR2A | MS4A4A | FCGR3A | MSR1 | C1QA |
| CYBB | FCER1G | PILRA | C5AR1 | RNASE6 | C1QC |
| FPR1 | LILRB4 | FCGR1A | CD86 | FGL2 | LILRB1 |
| MNDA | MS4A6A | FPR3 | IFI30 | LRRC25 | |
| HCK | CD14 | NCF2 | LYZ | FAM26F | |
| SPI1 | LST1 | MPEG1 | CD68 | C1orf162 | |
| CD163 | TYROBP | FCN1 | VSIG4 | C3AR1 | |

TABLE 3
IFNg Signature

| | |
|---|---|
| CCR5 | HLA-DRA |
| CXCL10 | IDO1 |
| CXCL11 | IFNG |
| CXCL9 | PRF1 |
| GZMA | STAT1 |

TABLE 4

| Reference | | | | | | Stages, Grade, |
|---|---|---|---|---|---|---|
| citation | Organism | Cell Types | Tissue | Disease | Indications | Metastasis |
| Lambrechts | Human | whole TME; | lung | NSCLC | NSCLC | non-metastatic |
| et al., | | cancer, | | | | |
| Nature | | stroma, | | | | |
| medicine | | immune, | | | | |
| 24.8 (2018): | | endothelial | | | | |
| 1277-1289. | | | | | | |
| Puram et | Human | whole TME; | oral cavity | head and | HNSCC | metastatic |
| al., Cell | | cancer, | | neck | | |
| 171.7 | | stroma, | | cancer | | |
| (2017): | | immune, | | | | |
| 1611-1624. | | endothelial | | | | |
| Tirosh et al., | Human | whole TME; | skin | melanoma | melanoma | metastatic |
| Science | | cancer, | | | | |
| 352.6282 | | stroma, | | | | |
| (2016): 189- | | immune, | | | | |
| 196. | | endothelial | | | | |
| Zheng et al., | Human | T cells | liver | HCC | HCC | unknown if met, |
| Cell 2017 | | | tumor, | | | stage I, II, IVB |
| Jun | | | peripheral | | | |
| 15; 169(7): | | | blood and | | | |
| 1342-1356.e16. | | | adjacent | | | |
| | | | normal | | | |
| | | | liver tissue | | | |
| Karaayvaz | Human | whole TME; | breast | TNBC | TNBC | non-metastatic |
| et al., | | cancer, | | | | |
| Nature | | stroma, | | | | |
| communications | | immune, | | | | |
| 9.1 (2018): | | endothelial | | | | |
| 1-10. | | (subset of | | | | |
| | | samples | | | | |
| | | underwent | | | | |
| | | CD45+ | | | | |
| | | depletion) | | | | |
| Sade- | Human | CD45+ | skin | melanoma | melanoma | metastatic |
| Feldman et | | immune cells | | | | |
| al., Cell | | | | | | |
| 175.4 | | | | | | |
| (2018): 998- | | | | | | |
| 1013. | | | | | | |
| Cillo et al., | Human | CD45+ | peripheral | HNSCC | HNSCC | unknown |
| Immunity 52.1 | | immune cells | blood and | | | |
| (2020): | | | tissue | | | |
| 183-199. | | | infiltration | | | |
| | | | immune | | | |
| | | | cells | | | |
| Qian et al., | Human | whole TME | ovarian, | ovarian, | | mixed stages |
| Cell Res | | | colorectal, | colorectal, | | |
| (2020). | | | lung, | lung, | | |
| https://doi. | | | breast | breast cancer | | |
| org/10.1038/ | | | | tumor and | | |
| s41422- | | | | adjacent | | |
| 020-0355-0 | | | | normals | | |

### Example 2

Analyses were carried out on patient samples in the selection of a dose for administration of JTX-8064. As is shown in Table 5, below, criteria tested included saturated target-mediated drug disposition (TMMD), which included measures of parallel elimination curves, stable clearance (CL) and half-life (T_{1/2}), and linear pharmacokinetics (PK); saturation of receptor occupancy (RO); and determination of Cmin >2× higher than lowest dose with saturation. A low dose meeting all the criteria was identified (700 mg) and selected for use as a dose for administration.

TABLE 5

| | | | Lowest dose |
|---|---|---|---|
| | | | meeting |
| Criterion | Measure | Met? | criterion |
| Saturated | Parallel elimination curves | ✓ | 300 mg |
| TMDD | Stable CL and T½ | ✓ | 300 mg |
| | Linear PK | ✓ | 50 mg |
| Saturated RO | | ✓ | 300 mg |

| | | |
|---|---|---|
| Cmin >2X higher than lowest dose with saturation | ✓ | 700 mg |

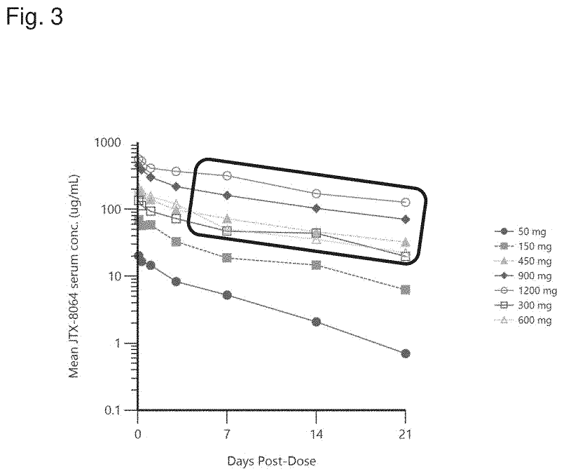

The log-linear concentration-time profile for JTX-8064 by dose is presented in FIG. 3. After IV administration, JTX-8064 is eliminated in a biphasic manner, and although there is some variability, elimination appears to be parallel at doses above 150 mg (i.e., TMDD appears to be saturated).

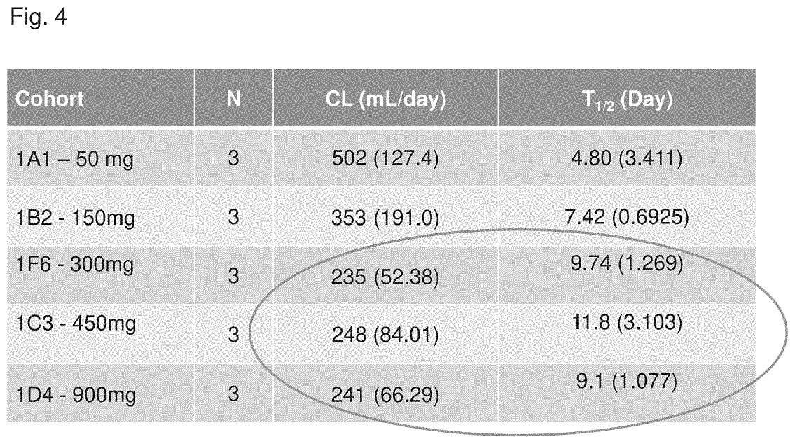

Mean (SD) clearance (CL) and T_{1/2} results are shown in FIG. 4. CL exhibits dose-dependent decrease and apparent elimination half-life (T_{1/2}) exhibits dose-dependent increases from 50 to 300 mg. Both parameters are stable and independent of dose starting at 300 mg, again consistent with full saturation of TMDD at the 300 mg dos

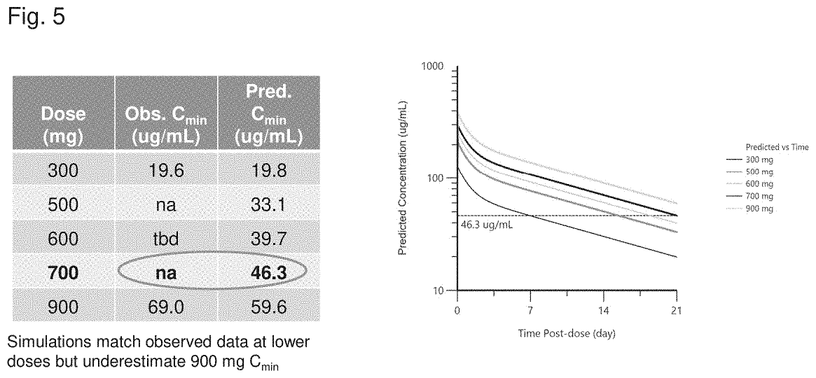

JTX-8064-concentration-time data were evaluated using standard compartmental modeling and found to fit a 2-compartment model FIG. 5. Mean parameters from a 2-compartment fit of the 300, 450, and 900 mg cycle 1 data were used to simulate PK for doses of 300 to 900 mg to identify a dose with a Cmin >2-fold above the Cmn at 300 mg. This margin was selected to ensure that patients at the low end of the exposure range would not be under-treated. The conservative >2-fold multiplier was selected taking into consideration the limited data currently available on the variability of JTX-8064 serum concentrations. The 700 mg dose results in a predicted Cmin of 46.3 ug/mL, which represents a 2.4-fold increase over the 19.6 ug/mL Cmin observed at the 300 mg dose.

### Example 3

### Target Dose Selection

Data on safety, preliminary JTX-8064 PK, and JTX-8064 receptor occupancy on monocytes (RO) have been utilized to determine the target dose, or preliminary recommended phase 2 dose (RP2D). The criteria for target dose selection are shown in Table.

TABLE 6
Target Dose Selection Criteria

| | | | Criterion |
|---|---|---|---|
| Criterion | Measure | Met? | met at: |
| Saturated | Parallel elimination curves | ✓ | ≥300 mg |
| TMDD | Stable clearance and half- | ✓ | ≥300 mg |
| | life | | |
| | Linear PK | ✓ | ≥50 mg |
| Saturated RO | No change in RO over | ✓ | ≥300 mg |
| | dosing interval | | |

| | | |
|---|---|---|
| Cmin >2X higher than the geometric mean at the | ✓ | ≥600 mg |
| lowest dose with saturated TMDD and RO | | |

Abbreviations:
Cmin—the minimum concentration in a dosing interval;
RO—receptor occupancy;
PK—pharmacokinetics;
TMDD—target-mediated drug disposition

The criterion for a Cmin greater than 2-fold higher than the Cmin at the first dose with saturated target-mediated drug disposition (TMDD) and LILRB2 receptor occupancy on circulating mononcytes (RO) was selected based on translational physiologically-based PK (PBPK) models suggesting tumor concentrations are at 2- to 3-fold lower than those in serum, which increases the likelihood of achieving tumor concentrations sufficient to saturate RO in the tumor microenvironment (TME).

The target dose was selected based on JTX-8064 PK and RO data. The 300 mg dose was identified as the lowest dose that achieved full saturation of RO across the dosing interval and also showed no TMDD based on non-compartmental analysis (NCA). The geometric mean Cycle 1 Day 21 (C1D21) Cₘᵢₙ (i.e. the concentration at the C2D1 pre-dose timepoint) at 300 mg was 19.6 μg/mL (n=3), with low interindividual variability (geometric coefficient of variation [GCV] of 19.5%). A target dose with the appropriate Cmin margin was selected based on simulations. Initially, to identify a dose with a Cmin close to 2.5-fold higher than this, observed JTX-8064 serum concentration-time data for doses of 300, 450, and 900 mg were fitted to a 2-compartment model and mean parameters determined. These parameters were used to simulate PK for additional doses of JTX-8064. Doses of 300, 450, 500, 600, 700, 750, 800 and 900 mg were simulated and Cycle 1 Cmin predicted. The 700 mg predicted Cmin of 46.3 μg/mL is 2.4-fold higher than the Cmin at 300 mg (19.6 μg/mL); this dose was therefore selected as the target dose.

### Example 4

### JTX-8064 Exposure Simulations

In order to identify the optimal biologically active dose under the maximum tolerated dose (MAD), first JTX-8064 exposures were simulated in a virtual population of subjects with solid tumors at dose levels of 300, 400, 500, 600, 700 and 800 mg Q3W. JTX-8064 was assumed to be administered as a 1-hour IV infusion once every three weeks (Q3W), and concentrations were simulated for the 1^{st} and 10^{th} cycles. The 10^{th} cycle concentrations were assumed to represent steady state.

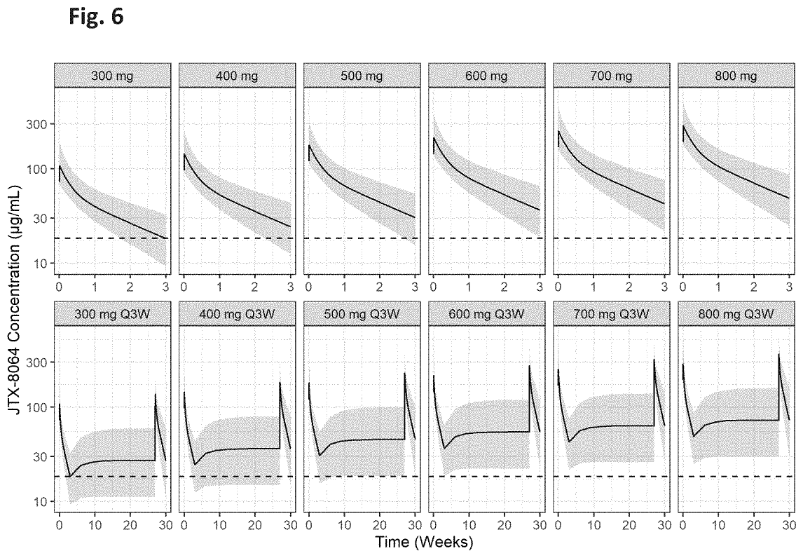

Body weight was sampled from a uniform distribution of body weights with upper and lower limits equal to the 5th and 95th percentiles of the subjects from the JTX-2011 analysis dataset (52 kg and 112 kg, respectively). The target Cₘᵢₙ concentration was defined as the median of the 300 mg predicted Cₘᵢₙ concentration range. Simulation results are shown graphically in FIG. 6.

Table 7 shows the numeric percentage of the subjects at a given dose anticipated to exceed the target concentration after single or multiple cycles.

TABLE 7
Percentage of Subjects Predicted to Exceed the Target
Concentration at Cmin After Single Dose (Cycle
1) or Steady State (Cycle 10) Administration

| | % of Subjects Achieving Target Concentration at Cₘᵢₙ | |
|---|---|---|
| Dosing Regimen | Cycle 1 | Cycle 10 |
| 300 mg Q3W | 50 | 79.3 |
| 400 mg Q3W | 77.9 | 90.9 |
| 500 mg Q3W | 90.2 | 95.2 |
| 600 mg Q3W | 95.1 | 97.7 |
| 700 mg Q3W | 97.5 | 98.7 |
| 800 mg Q3W | 98.7 | 99.2 |

Q3W: once every 3 weeks

The simulations demonstrate that >95% of subjects would achieve the target concentration after single doses of ≥600 mg, while at steady state this target would be achieved at doses of ≥500 mg.

TABLE 8
Cancer Therapies

| Anti-Cancer | | Anti-Cancer | |
|---|---|---|---|
| Therapeutic | Target Name | Therapeutic | Target Name |
| BMS-986179 | 5′-nucleotidase, ecto | imalumab | macrophage migration |
| | (CD73) | | inhibitory factor |
| | | | (glycosylation-inhibiting |
| | | | factor) |
| pTVG-HP | acid phosphatase, prostate | OSE-2101 | major histocompatibility |
| | | | complex, class I, A |
| sipuleucel-T | acid phosphatase, prostate | andecaliximab | matrix metallopeptidase 9 |
| | | | (gelatinase B, 92kDa |
| | | | gelatinase, 92kDa type IV |
| | | | collagenase) |
| CX-2009 | activated leukocyte cell | anti-MAGE-A3 | melanoma antigen family A, 3 |
| | adhesion molecule | TCR, Kite Pharma | |
| luspatercept | activin A receptor type II- | KITE-718 | melanoma antigen family A, 3 |
| | like 1 | | |
| CPI-444 | adenosine A2a receptor | biropepimut-S | melanoma antigen family A, 3 |
| NGR-TNF | alanyl (membrane) | rituximab | membrane-spanning 4- |
| | aminopeptidase | biosimilar, Pfizer | domains, subfamily A, |
| | | | member 1 |
| CB-1158 | arginase 1 | rituximab | membrane-spanning 4- |
| | arginase 2 | biosimilar, Dr. | domains, subfamily A, |
| | | Reddy's | member 1 |
| BA3011 | AXL receptor tyrosine | rituximab | membrane-spanning 4- |
| | kinase | biosimilar, Sandoz | domains, subfamily A, |
| | | | member 1 |
| AXL-107-MMAE | AXL receptor tyrosine | rituximab | membrane-spanning 4- |
| | kinase | biosimilar, Celltrion | domains, subfamily A, |
| | | | member 1 |
| CCT301-38 | AXL receptor tyrosine | rituximab | membrane-spanning 4- |
| | kinase | biosimilar, Archigen | domains, subfamily A, |
| | RAR-related orphan | Biotech | member 1 |
| | receptor A | | |
| SurVaxM | baculoviral IAP repeat | rituximab | membrane-spanning 4- |
| | containing 5 | biosimilar, Innovent | domains, subfamily A, |
| | | Biologies | member 1 |
| NY-ESO-1 TCR, | cancer/testis antigen 1 | MB-106 | membrane-spanning 4- |
| Adaptimmune | | | domains, subfamily A, |
| | | | member 1 |
| CDX-1401 | cancer/testis antigen 1 | ibritumomab | membrane-spanning 4- |
| | lymphocyte antigen 75 | tiuxetan | domains, subfamily A, |
| | | | member 1 |
| ETBX-011 | carcinoembryonic antigen- | rituximab | membrane-spanning 4- |
| | related cell adhesion | | domains, subfamily A, |
| | molecule 5 | | member 1 |
| GI-6207 | carcinoembryonic antigen- | ublituximab | membrane-spanning 4- |
| | related cell adhesion | | domains, subfamily A, |
| | molecule 5 | | member 1 |
| falimarev + | carcinoembryonic antigen- | rituximab | membrane-spanning 4- |
| inalimarev | related cell adhesion | biosimilar, | domains, subfamily A, |
| | molecule 5 | Allergan/Amgen | member 1 |
| | mucin 1, cell surface | | |
| | associated | | |
| labetuzumab | carcinoembryonic antigen- | ofatumumab | membrane-spanning 4- |
| govitecan | related cell adhesion | | domains, subfamily A, |
| | molecule 5 | | member 1 |
| | topoisomerase (DNA) I | | |
| coltuximab | CD19 molecule | ocaratuzumab | membrane-spanning 4- |
| ravtansine | | | domains, subfamily A, |
| | | | member 1 |
| denintuzumab | CD19 molecule | veltuzumab | membrane-spanning 4- |
| mafodotin | | | domains, subfamily A, |
| | | | member 1 |
| axicabtagene | CD19 molecule | obinutuzumab | membrane-spanning 4- |
| ciloleucel | | | domains, subfamily A, |
| | | | member 1 |
| CIK-CAR.CD19 | CD19 molecule | rituximab and | membrane-spanning 4- |
| | | hyaluronidase | domains, subfamily A, |
| | | human | member 1 |
| JCAR014 | CD19 molecule | anetumab | mesothelin |
| | | ravtansine | |
| lisocabtagene | CD19 molecule | amatuximab | mesothelin |
| maraleucel | | | |
| tisagenlecleucel | CD19 molecule | emibetuzumab | met proto-oncogene |
| MOR-208 | CD19 molecule | binimetinib | mitogen-activated protein |
| | | | kinase kinase 1 |
| | | | mitogen-activated protein |
| | | | kinase kinase 2 |
| inebilizumab | CD19 molecule | SAR566658 | mucin 1, cell surface |
| | | | associated |
| AUTO3, Autolus | CD19 molecule | Cvac, Prima | mucin 1, cell surface |
| | CD22 molecule | Biomed | associated |
| DT2219ARL | CD19 molecule | TG4010 | mucin 1, cell surface |
| | CD22 molecule | | associated |
| | | | interleukin 2 receptor, alpha |
| blinatumomab | CD19 molecule | oregovomab | mucin 16, cell surface |
| | CD3e molecule, epsilon | | associated |
| | (CD3-TCR complex) | | |
| samalizumab | CD200 molecule | methionine | opioid growth factor receptor |
| | | enkephalin based | |
| | | immunotherapy | |
| inotuzumab | CD22 molecule | olaratumab | platelet-derived growth factor |
| ozogamicin | | | receptor, alpha polypeptide |
| 90Y- | CD22 molecule | enfortumab vedotin | poliovirus receptor-related 4 |
| epratuzumab | | | |
| tetraxetan | | | |
| epratuzumab | CD22 molecule | ProstAtak, | polymerase (DNA directed), |
| | | Advantagene | alpha 1, catalytic subunit |
| ontuxizumab | CD248 molecule, | PancAtak, | polymerase (DNA directed), |
| | endosialin | Advantagene | alpha 1, catalytic subunit |
| varlilumab | CD27 molecule | aglatimagene | polymerase (DNA directed), |
| | | besadenovec | alpha 1, catalytic subunit |
| durvalumab | CD274 molecule | IMC-gp100 | premelanosome protein |
| avelumab | CD274 molecule | cemiplimab | programmed cell death 1 |
| atezolizumab | CD274 molecule | AGEN2034 | programmed cell death 1 |
| CX-072 | CD274 molecule | nivolumab | programmed cell death 1 |
| enoblituzumab | CD276 molecule | pembrolizumab | programmed cell death 1 |
| omburtamab | CD276 molecule | spartalizumab | programmed cell death 1 |
| AlloStim, | CD28 molecule | BGB-A317 | programmed cell death 1 |
| Immunovative | | | |
| Therapies | | | |
| gemtuzumab | CD33 molecule | genolimzumab | programmed cell death 1 |
| ozogamicin | | | |
| lintuzumab- | CD33 molecule | JNJ-63723283 | programmed cell death 1 |
| Ac225 | | | |
| BI 836858 | CD33 molecule | MEDI0680 | programmed cell death 1 |
| naratuximab | CD37 molecule | thymalfasin | prothymosin, alpha |
| emtansine | | | |
| lutetium (177Lu) | CD37 molecule | LYC-55716 | RAR-related orphan receptor |
| lilotomab | | | C |
| satetraxetan | | | |
| otlertuzumab | CD37 molecule | cirmtuzumab | receptor tyrosine kinase-like |
| | | | orphan receptor 1 |
| daratumumab | CD38 molecule | VX15/2503 | sema domain, |
| | | | immunoglobulin domain (Ig), |
| | | | transmembrane domain (TM) |
| | | | and short cytoplasmic |
| | | | domain, (semaphorin) 4D |
| isatuximab | CD38 molecule | elotuzumab | SLAM family member 7 |
| TAK-573 | CD38 molecule | indatuximab | syndecan 1 |
| | | ravtansine | |
| A-dmDT390- | CD3e molecule, epsilon | BMS-986207 | T-cell immunoreceptor with Ig |
| bisFv (UCHT1) | (CD3-TCR complex) | | and ITIM domains |
| APX005M | CD40 molecule, TNF | tertomotide | telomerase reverse |
| | receptor superfamily | | transcriptase |
| | member 5 | | |
| Hu5F9-G4 | CD47 molecule | Toca 511 + Toca | thymidylate synthetase |
| | | FC | |
| TI-061 | CD47 molecule | APS001F | thymidylate synthetase |
| milatuzumab | CD74 molecule, major | JCARH125 | TNF receptor superfamily |
| | histocompatibility complex, | | member 17 |
| | class II invariant chain | | |
| polatuzumab | CD79b molecule, | bb2121 | TNF receptor superfamily |
| vedotin | immunoglobulin-associated | | member 17 |
| | beta | | |
| mogamulizumab | chemokine (C-C motif) | AUTO2, Autolus | TNF receptor superfamily |
| | receptor 4 | | member 17 |
| | | | TNF receptor superfamily |
| | | | member 13B |
| BL-8040 | chemokine (C-X-C motif) | OPN-305 | toll-like receptor 2 |
| | receptor 4 | | |
| X4P-001 | chemokine (C-X-C motif) | rintatolimod | toll-like receptor 3 |
| | receptor 4 | | |
| ulocuplumab | chemokine (C-X-C motif) | poly-ICLC | toll-like receptor 3 |
| | receptor 4 | | |
| claudiximab | claudin 18 | ID-G100 | toll-like receptor 4 |
| ALT-836 | coagulation factor III | ID-CMB305 | toll-like receptor 4 |
| | (thromboplastin, tissue | | cancer/testis antigen 1 |
| | factor) | | |
| MCS110 | colony stimulating factor 1 | imiquimod | toll-like receptor 7 |
| | (macrophage) | (intravesical), | |
| | | Telormedix | |
| ARRY-382 | colony stimulating factor 1 | NKTR-262 | toll-like receptor 7 |
| | (macrophage) | | toll-like receptor 8 |
| | colony stimulating factor 1 | | |
| | receptor | | |
| BLZ-945 | colony stimulating factor 1 | motolimod | toll-like receptor 8 |
| | receptor | | |
| AMG 820 | colony stimulating factor 1 | tilsotolimod | toll-like receptor 9 |
| | receptor | | |
| cabiralizumab | colony stimulating factor 1 | sacituzumab | topoisomerase (DNA) I |
| | receptor | govitecan | tumor-associated calcium |
| | | | signal transducer 2 |
| gemogenovatucel-T | colony stimulating factor 2 | HPV-16 E6 TCR, | transforming protein E6, |
| | (granulocyte-macrophage) | Bluebird Bio/Kite | human papilloma virus-16 |
| | | Pharma | |
| GVAX | colony stimulating factor 2 | VGX-3100 | transforming protein E6, |
| | (granulocyte-macrophage) | | human papilloma virus-16 |
| | | | transforming protein E7, |
| | | | human papilloma virus-16 |
| | | | E6 protein, human papilloma |
| | | | virus-18 |
| | | | E7 protein, human papilloma |
| | | | virus-18 |
| talimogene | colony stimulating factor 2 | MEDI0457 | transforming protein E6, |
| laherparepvec | (granulocyte-macrophage) | | human papilloma virus-16 |
| | | | transforming protein E7, |
| | | | human papilloma virus-16 |
| | | | E7 protein, human papilloma |
| | | | virus-18 |
| | | | E6 protein, human papilloma |
| | | | virus-18 |
| pexastimogene | colony stimulating factor 2 | TVGV-1 | transforming protein E7, |
| devacirepvec | (granulocyte-macrophage) | | human papilloma virus-16 |
| sargramostim | colony stimulating factor 2 | KITE-439 | transforming protein E7, |
| | receptor, alpha, low-affinity | | human papilloma virus-16 |
| | (granulocyte-macrophage) | | |
| SV-BR-1-GM | colony stimulating factor 2 | ADXS-DUAL | transforming protein E7, |
| cancer vaccine | receptor, alpha, low-affinity | | human papilloma virus-16 |
| | (granulocyte-macrophage) | | |
| pamrevlumab | connective tissue growth | axalimogene | transforming protein E7, |
| | factor | filolisbac | human papilloma virus-16 |
| ipilimumab | cytotoxic T-lymphocyte- | MVA-5T4 | trophoblast glycoprotein |
| | associated protein 4 | | |
| tremelimumab | cytotoxic T-lymphocyte- | oportuzumab | tumor-associated calcium |
| | associated protein 4 | monatox | signal transducer 2 |
| BMS-986249 | cytotoxic T-lymphocyte- | denosumab | tumour necrosis factor |
| | associated protein 4 | | (ligand) superfamily, member |
| | | | 11 |
| rovalpituzumab | delta-like 3 (Drosophila) | BION-1301 | tumour necrosis factor |
| tesirine | | | (ligand) superfamily, member |
| | | | 13 |
| ABT-165 | delta-like 4 (Drosophila) | belimumab | tumour necrosis factor |
| | vascular endothelial growth | | (ligand) superfamily, member |
| | factor A | | 13b |
| BHQ880 | dickkopf WNT signaling | INCAGN1876 | tumour necrosis factor |
| | pathway inhibitor 1 | | receptor superfamily, member |
| | | | 18 |
| DKN-01 | dickkopf WNT signaling | BMS-986156 | tumour necrosis factor |
| | pathway inhibitor 1 | | receptor superfamily, member |
| | | | 18 |
| Ad-REIC vaccine, | dickkopf WNT signaling | INCAGN1949 | tumour necrosis factor |
| Momotaro-Gene | pathway inhibitor 3 | | receptor superfamily, member |
| | | | 4 |
| AGS-16C3F | ectonucleotide | PF-04518600 | tumour necrosis factor |
| | pyrophosphatase/phosphodi- | | receptor superfamily, member |
| | esterase 3 | | 4 |
| carotuximab | endoglin | BMS-986178 | tumour necrosis factor |
| | | | receptor superfamily, member |
| | | | 4 |
| ifabotuzumab | EPH receptor A3 | brentuximab | tumour necrosis factor |
| | | vedotin | receptor superfamily, member |
| | | | 8 |
| CimaVax EGF | epidermal growth factor | urelumab | tumour necrosis factor |
| | (beta-urogastrone) | | receptor superfamily, member |
| | | | 9 |
| depatuxizumab | epidermal growth factor | utomilumab | tumour necrosis factor |
| mafodotin | receptor | | receptor superfamily, member |
| | | | 9 |
| RM-1929 | epidermal growth factor | VBI-1901 | UL83, cytomegalovirus |
| | receptor | | UL55, cytomegalovirus |
| AVID100 | epidermal growth factor | bevacizumab | vascular endothelial growth |
| | receptor | biosimilar, | factor A |
| | | Boehringer | |
| | | Ingelheim | |
| trastuzumab | epidermal growth factor | bevacizumab-awwb | vascular endothelial growth |
| biosimilar, | receptor | | factor A |
| Henlius | | | |
| cetuximab | epidermal growth factor | bevacizumab | vascular endothelial growth |
| | receptor | biosimilar, Pfizer | factor A |
| panitumumab | epidermal growth factor | bevacizumab | vascular endothelial growth |
| | receptor | biosimilar, | factor A |
| | | Oncobiologics | |
| necitumumab | epidermal growth factor | bevacizumab | vascular endothelial growth |
| | receptor | biosimilar, Henlius | factor A |
| | | Biopharmaceuticals | |
| nimotuzumab | epidermal growth factor | bevacizumab | vascular endothelial growth |
| | receptor | biosimilar, Fujifilm | factor A |
| | | Kyowa Kirin | |
| | | Biologies | |
| futuximab | epidermal growth factor | aflibercept | vascular endothelial growth |
| | receptor | | factor A |
| tomuzotuximab | epidermal growth factor | bevacizumab | vascular endothelial growth |
| | receptor | | factor A |
| doxorubicin, EDV | epidermal growth factor | pritumumab | vimentin |
| nanocells, | receptor | | |
| EnGeneIC | | | |
| pan-HER | epidermal growth factor | pexidartinib | v-kit Hardy-Zuckerman 4 |
| | receptor | | feline sarcoma viral oncogene |
| | erb-b2 receptor tyrosine | | homologue |
| | kinase 2 | | colony stimulating factor 1 |
| | erb-b2 receptor tyrosine | | receptor |
| | kinase 3 | | fms-related tyrosine kinase 3 |
| trastuzumab | erb-b2 receptor tyrosine | galinpepimut-S | Wilms tumour 1 |
| deruxtecan | kinase 2 | | |
| trastuzumab | erb-b2 receptor tyrosine | adegramotide/ | Wilms tumour 1 |
| emtansine | kinase 2 | nelatimotide | |
| (vic-)trastuzumab | erb-b2 receptor tyrosine | JTCR016 | Wilms tumour 1 |
| duocarmazine | kinase 2 | | |
| nelipepimut-S | erb-b2 receptor tyrosine | levamisole | Unknown |
| | kinase 2 | | |
| trastuzumab | erb-b2 receptor tyrosine | ladiratuzumab | Unknown |
| biosimilar, Merck | kinase 2 | vedotin | |
| & Co./Samsung | | | |
| Bioepis | | | |
| trastuzumab | erb-b2 receptor tyrosine | NSC-631570 | Unknown |
| biosimilar, | kinase 2 | | |
| Celltrion | | | |
| trastuzumab | erb-b2 receptor tyrosine | LN-145 | Unknown |
| biosimilar, Biocon | kinase 2 | | |
| trastuzumab | erb-b2 receptor tyrosine | INO-5401 | Unknown |
| biosimilar, | kinase 2 | | |
| Allergan/Amgen | | | |
| trastuzumab | erb-b2 receptor tyrosine | AN01, Anson | Unknown |
| biosimilar, Pfizer | kinase 2 | Pharma | |
| AU101, Aurora | erb-b2 receptor tyrosine | GALE-302 | Unknown |
| Biopharma | kinase 2 | | |
| AU105, Aurora | erb-b2 receptor tyrosine | MAGE-A3 TCR, | Unknown |
| BioPharma | kinase 2 | Adaptimmune | |
| AE37 | erb-b2 receptor tyrosine | BTH-1677 | Unknown |
| | kinase 2 | | |
| trastuzumab | erb-b2 receptor tyrosine | lentinan | Unknown |
| | kinase 2 | | |
| pertuzumab | erb-b2 receptor tyrosine | Polysaccharide-K | Unknown |
| | kinase 2 | | |
| margetuximab | erb-b2 receptor tyrosine | Tice BCG, Organon | Unknown |
| | kinase 2 | | |
| ADXS31-164 | erb-b2 receptor tyrosine | IGEM-F | Unknown |
| | kinase 2 | | |
| ETBX-021 | erb-b2 receptor tyrosine | PV-10, Provectus | Unknown |
| | kinase 2 | | |
| seribantumab | erb-b2 receptor tyrosine | vitespen | Unknown |
| | kinase 3 | | |
| patritumab | erb-b2 receptor tyrosine | mifamurtide | Unknown |
| | kinase 3 | | |
| CDX-3379 | erb-b2 receptor tyrosine | melanoma vaccine, | Unknown |
| | kinase 3 | GSK | |
| elgemtumab | erb-b2 receptor tyrosine | Bacille Calmette- | Unknown |
| | kinase 3 | Guerin vaccine, ID | |
| | | Biomedical | |
| moxetumomab | eukaryotic translation | seviprotimut-l | Unknown |
| pasudotox | elongation factor 2 | | |
| | CD22 molecule | | |
| denileukin diftitox | eukaryotic translation | in situ autologous | Unknown |
| | elongation factor 2 | cancer vaccine, | |
| | interleukin 2 receptor, alpha | Immunophotonics | |
| MDNA55 | eukaryotic translation | IMA901 | Unknown |
| | elongation factor 2 | | |
| | interleukin 4 receptor | | |
| bemarituzumab | fibroblast growth factor | adagloxad | Unknown |
| | receptor 2 | simolenin | |
| DCVax-prostate, | folate hydrolase (prostate- | PVX-410 | Unknown |
| Northwest | specific membrane antigen) | | |
| Biotherapeutics | 1 | | |
| 177Lu-J591 | folate hydrolase (prostate- | viagenpumatucel-L | Unknown |
| | specific membrane antigen) | | |
| | 1 | | |
| tuberculosis | folate hydrolase (prostate- | GALE-301 | Unknown |
| vaccine (Mw), | specific membrane antigen) | | |
| Cadila; Cadi-05 | 1 | | |
| mirvetuximab | folate receptor 1 (adult) | EP-302, EpiThany | Unknown |
| soravtansine | | | |
| TPIV200 | folate receptor 1 (adult) | BI 1361849 | Unknown |
| farletuzumab | folate receptor 1 (adult) | DPV-001 | Unknown |
| IGEM-FR | folate receptor 1 (adult) | Bacille Calmette- | Unknown |
| | | Guerin vaccine, | |
| | | Sanofi | |
| G17DT | gastrin | LAMP-Vax + pp65 | Unknown |
| | | DC, Immunomic | |
| | | Therapeutics | |
| codrituzumab | glypican 3 | NKG2D-CAR | Unknown |
| EP-100, | gonadotropin-releasing | BPX-501 | Unknown |
| EpiThany | hormone 1 (luteinizing- | | |
| | releasing hormone) | | |
| | luteinizing | | |
| | hormone/choriogonadotropin | | |
| | receptor | | |
| naxitamab | growth differentiation factor | NK-92 cells | Unknown |
| | 2 | | |
| CDX-014 | hepatitis A virus cellular | LN-144 | Unknown |
| | receptor 1 | | |
| MBG453 | hepatitis A virus cellular | CLBS-23 | Unknown |
| | receptor 2 | | |
| histamine | histamine receptor H2 | DCVax-Direct, | Unknown |
| dihydrochloride | | Northwest | |
| | | Biotherapeutics | |
| entinostat | histone deacetylase 1 | melanoma vaccine, | Unknown |
| | | AVAX | |
| indoximod | indoleamine-pyrrole 2,3 | stapuldencel-T | Unknown |
| | dioxygenase | | |
| epacadostat | indoleamine-pyrrole 2,3 | dendritic cancer | Unknown |
| | dioxygenase | vaccine, DanDrit | |
| | | Biotech | |
| BMS-986205 | indoleamine-pyrrole 2,3 | DCVax-Brain | Unknown |
| | dioxygenase | brain cancer | |
| | | vaccine, Northwest | |
| | | Biotherapeutics | |
| JTX-2011 | inducible T-cell co- | tumor lysate | Unknown |
| | stimulator | particle-loaded | |
| | | dendritic cell | |
| | | vaccine, Perseus | |
| BMS-986226 | inducible T-cell co- | ERC1671 | Unknown |
| | stimulator | | |
| ADC W0101 | insulin-like growth factor 1 | BSK01 | Unknown |
| | receptor | TAPA pulsed DC | |
| | | vaccine | |
| ganitumab | insulin-like growth factor 1 | Oncoquest-CLL | Unknown |
| | receptor | vaccine | |
| istiratumab | insulin-like growth factor 1 | rocapuldencel-T | Unknown |
| | receptor | | |
| | erb-b2 receptor tyrosine | | |
| | kinase 3 | | |
| dusigitumab | insulin-like growth factor 1 | ATIR-101 | Unknown |
| | receptor | | |
| | insulin-like growth factor 2 | | |
| | receptor | | |
| EP-201, | insulin-like growth factor | TVI-Kidney-1 | Unknown |
| EpiThany | binding protein 2, 36kDa | | |
| citoplurikin | interferon gamma receptor | TVAX cancer | Unknown |
| | 1 | vaccine, TVAX | |
| | tumour necrosis factor | Biomedical | |
| | receptor superfamily, | | |
| | member 1A | | |
| MABp1 | interleukin 1, alpha | atezolizumab, | Unknown |
| | | companion | |
| | | diagnostic | |
| pegilodecakin | interleukin 10 | tumour infiltrating | Unknown |
| | | lymphocytes, | |
| | | lovance | |
| | | Biotherapeutics-2 | |
| Ad-RTS-hIL-12 + | interleukin 12 receptor, | MAGE A-10 TCR, | Unknown |
| veledimex | beta 1 | Adaptimmune | |
| tavokinogene | interleukin 12 receptor, | IMA101 | Unknown |
| telsaplasmid | beta 1 | | |
| | interleukin 12 receptor, | | |
| | beta 2 | | |
| EGEN-001 | interleukin 12A (natural | algenpantucel-L | Unknown |
| | killer cell stimulatory factor | | |
| | 1, cytotoxic lymphocyte | | |
| | maturation factor 1, p35) | | |
| | interleukin 12B (natural | | |
| | killer cell stimulatory factor | | |
| | 2, cytotoxic lymphocyte | | |
| | maturation factor 2, p40) | | |
| SL-701 | interleukin 13 receptor, | Tumor Necrosis | Unknown |
| | alpha 2 | Therapy, Peregrine | |
| | EPH receptor A2 | | |
| | baculoviral IAP repeat | | |
| | containing 5 | | |
| ALT-803 | interleukin 15 receptor, | imiquimod | Unknown |
| | alpha | | |
| Multikine, Cel-Sci | interleukin 2 receptor, alpha | LOAd703 | Unknown |
| ALT-801 | interleukin 2 receptor, alpha | CG0070 | Unknown |
| high-affinity | interleukin 2 receptor, alpha | dinutuximab | Unknown |
| Natural Killer | | | |
| (haNK) cells, | | | |
| NantKwest | | | |
| interleukin-2, | interleukin 2 receptor, alpha | bavituximab | Unknown |
| Roche | | | |
| aldesleukin | interleukin 2 receptor, alpha | ensituximab | Unknown |
| NKTR-214 | interleukin 2 receptor, beta | pidilizumab | Unknown |
| talacotuzumab | interleukin 3 receptor, alpha | BMS-986218 | Unknown |
| | (low affinity) | | |
| SL-401 | interleukin 3 receptor, alpha | BMS-986012 | Unknown |
| | (low affinity) | | |
| siltuximab | interleukin 6 (interferon, | ADXS31-142 | Unknown |
| | beta 2) | | |
| HuMax-IL8 | interleukin 8 | GI-6301 | Unknown |
| PSA/IL-2/GM- | kallikrein-related peptidase | GI-4000 | Unknown |
| CSF | 3 | | |
| rilimogene | kallikrein-related peptidase | JNJ-64041757 | Unknown |
| galvacirepvec | 3 | | |
| | CD80 molecule | | |
| | intercellular adhesion | | |
| | molecule 1 | | |
| | CD58 molecule | | |
| monalizumab | killer cell lectin-like receptor | HPV vaccine | Unknown |
| | subfamily C, member 1 | (Cervarix), GSK | |
| ramucirumab | kinase insert domain | HPV vaccine | Unknown |
| | receptor | (Gardasil), CSL | |
| ubenimex | leucotriene A4 hydrolase | Sym015 | Unknown |
| | leucotriene B4 receptor | | |
| IMP321 | lymphocyte-activation gene | diphenyl- | Unknown |
| | 3 | cyclopropenone | |
| LAG525 | lymphocyte-activation gene | ISA101 | Unknown |
| | 3 | | |
| relatlimab | lymphocyte-activation gene | | |
| | 3 | | |

TABLE 9
Sequences-JTX-8064

| SEQ | | |
|---|---|---|
| ID NO: | Description | Sequence |
| 1 | J-19.h1 | QITLKESGPTLVKPTQTLTLTCTF |
| | heavy chain | SGFSLNTYAMGVSWIRQPPGKALE |
| | | WLASIWWNGNKYNNPSLKSRLTVT |
| | | KDTSKNQVVLTMTNMDPVDTATYY |
| | | CAHSRIIRFTDYVMDAWGQGTLVT |
| | | VSSASTKGPSVFPLAPCSRSTSES |
| | | TAALGCLVKDYFPEPVTVSWNSGA |
| | | LTSGVHTFPAVLQSSGLYSLSSVV |
| | | TVPSSSLGTKTYTCNVDHKPSNTK |
| | | VDKRVESKYGPPCPPCPAPEFLGG |
| | | PSVFLFPPKPKDTLMISRTPEVTC |
| | | VVVDVSQEDPEVQFNWYVDGVEVH |
| | | NAKTKPREEQFNSTYRVVSVLTVL |
| | | HQDWLNGKEYKCKVSNKGLPSSIE |
| | | KTISKAKGQPREPQVYTLPPSQEE |
| | | MTKNQVSLTCLVKGFYPSDIAVEW |
| | | ESNGQPENNYKTTPPVLDSDGSFF |
| | | LYSRLTVDKSRWQEGNVFSCSVMH |
| | | EALHNHYTQKSLSLSLG |
| 2 | J-19.h1 | DIQMTQSPSSLSTSVGDRVTITCR |
| | light chain | ASEDIYNDLAWYQQKPGKAPKLLI |
| | | YNANSLHTGVASRFSGSGSGTDFT |
| | | FTISSLQPEDVATYFCQQYYDYPL |
| | | TFGQGTKLEIKRTVAAPSVFIFPP |
| | | SDEQLKSGTASVVCLLNNFYPREA |
| | | KVQWKVDNALQSGNSQESVTEQDS |
| | | KDSTYSLSSTLTLSKADYEKHKVY |
| | | ACEVTHQGLSSPVTKSFNRGEC |
| 3 | J-19.h1 V_{H} | QITLKESGPTLVKPTQTLTLTCTF |
| | | SGFSLNTYAMGVSWIRQPPGKALE |
| | | WLASIWWNGNKYNNPSLKSRLTVT |
| | | KDTSKNQVVLTMTNMDPVDTATYY |
| | | CAHSRIIRFTDYVMDAWGQGTLVT |
| | | VSS |
| 4 | J-19.h1 V_{L} | DIQMTQSPSSLSTSVGDRVTITCR |
| | | ASEDIYNDLAWYQQKPGKAPKLLI |
| | | YNANSLHTGVASRFSGSGSGTDFT |
| | | FTISSLQPEDVATYFCQQYYDYPL |
| | | TFGQGTKLEIK |
| 5 | J-19.h1 CDR-H1 | TYAMGVS |
| 6 | J-19.h1 CDR-H2 | SIWWNGNKYNNPSLKS |
| 7 | J-19.h1 CDR-H3 | SRIIRFTDYVMDA |
| 8 | J-19.h1 CDR-L1 | RASEDIYNDLA |
| 9 | J-19.h1 CDR-L2 | NANSLHT |
| 10 | J-19.h1 CDR-L3 | QQYYDYPLT |

Other embodiments are within the scope of the following claims.

What is claimed is:

1. A method of treating cancer in a subject, the method comprising determining the ratio of LILRB2 to IFNg in a sample from the subject and, if elevated LILRB2 relative to IFNg is detected, administering a LILRB2 antibody and a PD1 antagonist to the subject.

2. A method of treating cancer in a subject, the method comprising determining the ratio of LILRB2 to IFNg in a sample from the subject and, if a balanced level of LILRB2 and IFNg, or elevated IFNg relative to LILRB2, is detected, administering a PD1 antagonist to the subject in the absence of a LILRB2 antibody.

3. A method for treating cancer in a subject, the method comprising administering a LILRB2 antibody and a PD1 antagonist to the subject, wherein elevated LILRB2 relative to IFNg has been detected in a sample from the subject.

4. A method of treating cancer in a subject, the method comprising administering a PD1 antagonist to the subject, in the absence of a LILRB2 antibody, wherein a balanced level of LILRB2 and IFNg, or elevated IFNg relative to LILRB2, has been detected in a sample from the subject.

5. A method for identifying a subject whose cancer is likely to have an improved response to combination therapy with a LILRB2 antibody and a PD1 antagonist, the method comprising determining the ratio of LILRB2 to IFNg in a sample from the subject, wherein detection of elevated LILRB2 relative to IFNg indicates that a subject is likely to have an improved response to the combination therapy.

6. A method for identifying a subject whose cancer is likely to respond to a PD1 antagonist, without improvement by combination treatment with a LILRB2 antibody, the method comprising determining the ratio of LILRB2 to IFNg in a sample from the subject, wherein detection of a balanced level of LILRB2 and IFNg, or elevated IFNg relative to LILRB2, indicates that a subject is likely to respond to a PD1 antagonist, without improvement by combination treatment with a LILRB2 antibody.

7. A method for selecting a cancer therapy for a subject, the method comprising determining the ratio of LILRB2 to IFNg in a sample from the subject, wherein detection of elevated LILRB2 relative to IFNg indicates selection of a LILRB2 antibody and a PD1 antagonist for treatment of the subject.

8. A method for selecting a cancer therapy for a subject, the method comprising determining the ratio of LILRB2 to IFNg in a sample from the subject, wherein detection of a balanced level of LILRB2 and IFNg, or elevated IFNg relative to LILRB2, indicates selection of a PD1 antagonist for treatment of the subject, in the absence of a LILRB2 antibody.

9. A method of improving the response of a subject to PD1 antagonist cancer therapy, the method comprising administering a LILRB2 antibody to the subject, wherein elevated LILRB2 relative to IFNg has been detected in a sample from the subject.

10. The method of claim 5 or 7, further comprising administering a LILRB2 antibody and a PD1 antagonist to the subject.

11. The method of claim 6 or 8, further comprising administering a PD1 antagonist to the subject, in the absence of a LILRB2 antibody.

12. The method of claim 9, further comprising administering a PD1 antagonist to the subject.

13. The method of any one of claim 1, 3, 10, or 12, wherein the therapy comprises administration of the LILRB2 antibody and the PD1 antagonist at about the same time as one another.

14. The method of any one of claim 1, 3, 10, or 12, wherein the combination therapy comprises administration of the LILRB2 antibody before the PD1 antagonist.

15. The method of any one of claims 1 to 14, wherein detection of LILRB2 and/or IFNg levels is carried out by detection of LILRB2 and/or IFNg RNA levels.

16. The method of any one of claims 1 to 15, wherein detection of LILRB2 and/or IFNg levels is carried out by detection of LILRB2 and/or IFNg protein levels.

17. The method of any one of claims 1 to 16, wherein detection of LILRB2 and/or IFNg levels is carried out by detection of a LILRB2 signature, which optionally is a tumor-associated macrophage (TAM) gene signature, and/or IFNg gene signature.

18. The method of any one of claims 1 to 17, wherein the sample comprises a tumor biopsy.

19. The method of any one of claims 1 to 18, wherein the LILRB2 antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

20. The method of claim 19, wherein the antibody comprises a V_{H} region comprising an amino acid sequence that is at least 90% identical to the amino acid sequence of SEQ ID NO: 3 and a V_{L} region comprising an amino acid sequence that is at least 90% identical to the amino acid sequence of SEQ ID NO: 4, wherein the V_{H} region comprises three CDRs comprising the amino acid sequences of SEQ ID NOs: 5-7, and the V_{L} region comprises three CDRs comprising the amino acid sequences of SEQ ID NOs: 8-10.

21. The method of claim 19 or 20, wherein the antibody comprises a V_{H} region comprising the amino acid sequence of SEQ ID NO: 3 and a variable light chain V_{L} region comprising the amino acid sequence of SEQ ID NO: 4.

22. The method of any one of claims 19 to 21, wherein the antibody comprises a heavy chain comprising the amino acid sequence of SEQ ID NO: 1 and a light chain comprising the amino acid sequence of SEQ ID NO: 2.

23. The method of any one of claims 1 to 18, wherein the LILRB2 antibody is selected from the group consisting of: JTX-8064, MK-4830, NGM707, IO-108, and iosH2.

24. The method of any one of claims 1 to 23, wherein the PD1 antagonist is directed against PD1.

25. The method of any one of claims 1 to 23, wherein the PD1 antagonist is directed against PD-L1.

26. The method of any one of claims 1 to 25, wherein the PD1 antagonist comprises an antibody.

27. The method of claim 26, wherein the PD1 antagonist antibody is selected from the group consisting of: JTX-4014, nivolumab, pidilizumab, lambrolizumab, pembrolizumab, cemiplimab, avelumab, atezolizumab tislelizumab, durvalumab, spartalizumab, genolimzumab, BGB-A317, RG-7446, AMP-224, BMS-936559, AMP-514, MDX-1105, AB-011, RG7446/MPDL3280A, KD-033, AGEN-2034, STI-A1010, STI-A1110, TSR-042, CX-072, JNJ-63723283, and JNC-1.

28. The method of claim 27, wherein the PD1 antagonist antibody is JTX-4014.

29. The method of any one of claims 1 to 28, wherein the cancer of the subject is selected from the group consisting of gastric cancer, melanoma (e.g., skin cutaneous melanoma), urothelial cancer, lymphoid neoplasm, diffuse large B-cell lymphoma (DLBCL), testicular germ cell tumors (TGCT), mesothelioma, kidney cancer (e.g., kidney renal clear cell carcinoma, kidney renal papillary cell carcinoma, or renal cell carcinoma (RCC)), sarcoma, lung cancer (e.g., lung adenocarcinoma, lung squamous cell carcinoma, and non-small cell lung cancer (NSCLC)) stomach adenocarcinoma, pancreatic adenocarcinoma, head and neck squamous cell carcinoma, ovarian serious cystadenocarcinoma, liver hepatocellular carcinoma, skin cutaneous melanoma, colon adenocarcinoma, breast cancer (e.g., breast invasive carcinoma or triple negative breast cancer), rectum adenocarcinoma, glioblastoma multiforme, uterine corpus endometrial carcinoma, thymoma, bladder cancer, endometrial cancer, Hodgkin's lymphoma, ovarian cancer, anal cancer, biliary cancer, colorectal cancer, and esophageal cancer.

30. The method of claim 29, wherein the cancer of the subject is gastric cancer, melanoma, or urothelial cancer.

31. The method of any one of claims 1 to 4 or 9 to 30, further comprising administration of an additional therapeutic agent to the subject.

32. A kit for use in determining whether to administer a combination of a LILRB2 antibody and a PD1 antagonist to a subject having cancer according to a method described herein, the kit comprising primers, probes, and/or antibodies for detecting the level of LILRB2 RNA or protein, IFNg RNA or protein, and/or the components of a gene signature for LILRB2 and/or IFNg in a sample from the subject.

33. A composition comprising a unit dose of an antibody that specifically binds to human LILRB2, wherein the unit dose is 300 to 1200 mg, e.g., 400 to 900 mg or 450 to 900 mg, and the antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

34. The composition of claim 33, wherein the antibody comprises a V_{H} region comprising an amino acid sequence that is at least 90% identical to the amino acid sequence of SEQ ID NO: 3 and a V_{L} region comprising an amino acid sequence that is at least 90% identical to the amino acid sequence of SEQ ID NO: 4, wherein the V_{H} region comprises three CDRs comprising the amino acid sequences of SEQ ID NOs: 5-7, and the V_{L} region comprises three CDRs comprising the amino acid sequences of SEQ ID NOs: 8-10.

35. The composition of claim 33 or 34, wherein the antibody comprises a V_{H} region comprising the amino acid sequence of SEQ ID NO: 3 and a variable light chain V_{L} region comprising the amino acid sequence of SEQ ID NO: 4.

36. The composition of any one of claims 33 to 35, wherein the antibody comprises a heavy chain comprising the amino acid sequence of SEQ ID NO: 1 and a light chain comprising the amino acid sequence of SEQ ID NO: 2.

37. The composition of any one of claims 33 to 36, wherein the dose is within a range selected from the group consisting of: 300-450 mg, 350-500 mg, 400-550 mg, 450-600 mg, 500-650 mg, 550-700 mg, 600-750 mg, 650-800 mg, 700-850 mg, 750-900 mg, 800-950 mg, 850-1000 mg, 900-1050 mg, and 950-1200 mg.

38. The composition of any one of claims 33 to 36, wherein the dose is within a range selected from the group consisting of: 300-400 mg, 350-450 mg, 400-500 mg, 450-550 mg, 500-600 mg, 550-650 mg, 600-700 mg, 650-750 mg, 700-800 mg, 750-850 mg, 800-900 mg, 850-950 mg, 900-1000 mg, 950-1050 mg, 1000-1100 mg, 1050-1150 mg, and 1100-1200 mg.

39. The composition of any one of claims 33 to 38, wherein the dose is selected from the group consisting of 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, and 1200 mg.

40. The composition of any one of claims 33 to 39, wherein the dose is within the range of 600-800 mg.

41. The composition of any one of claims 33 to 40, wherein the dose is 700 mg.

42. A method of treating cancer in a subject, the method comprising administering an antibody that specifically binds to LILRB2 to the subject at a dose is 300 to 1200 mg, e.g., 400 to 900 mg or 450 to 900 mg, wherein the antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

43. The method of claim 42, wherein the dose is within a range selected from the group consisting of: 300-450 mg, 350-500 mg, 400-550 mg, 450-600 mg, 500-650 mg, 550-700 mg, 600-750 mg, 650-800 mg, 700-850 mg, 750-900 mg, 800-950 mg, 850-1000 mg, 900-1050 mg, and 950-1200 mg.

44. The method of claim 42, wherein the dose is within a range selected from the group consisting of: 300-400 mg, 350-450 mg, 400-500 mg, 450-550 mg, 500-600 mg, 550-650 mg, 600-700 mg, 650-750 mg, 700-800 mg, 750-850 mg, 800-900 mg, 850-950 mg, 900-1000 mg, 950-1050 mg, 1000-1100 mg, 1050-1150 mg, and 1100-1200 mg.

45. The method of any one of claims 42 to 44, wherein the dose is selected from the group consisting of 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, and 1200 mg.

46. The method of any one of claims 42 to 45, wherein the dose is within the range of 600-800 mg.

47. The method of any one of claims 42 to 46, wherein the dose is 700 mg.

48. A method of treating cancer in a subject, the method comprising administering an antibody that specifically binds to LILRB2 to the subject at a dose of 5-15 mg/kg, wherein the antibody comprises the following six complementarity determining regions (CDRs):
(a) a CDR-H1 comprising the amino acid sequence of SEQ ID NO: 5;
(b) a CDR-H2 comprising the amino acid sequence of SEQ ID NO: 6;
(c) a CDR-H3 comprising the amino acid sequence of SEQ ID NO: 7;
(d) a CDR-L1 comprising the amino acid sequence of SEQ ID NO: 8;
(e) a CDR-L2 comprising the amino acid sequence of SEQ ID NO: 9; and
(f) a CDR-L3 comprising the amino acid sequence of SEQ ID NO: 10.

49. The method of claim 48, wherein the dose is within a range selected from the group consisting of: 5-10 mg/kg, 7.5-12.5 mg/kg, and 10-15 mg/kg.

50. The method of any one of claims 42 to 49, wherein a dose of the antibody is administered once every three weeks.

51. The method of claim 50, comprising administering the antibody in said dose, once every three weeks, for 1-12 cycles.

52. The method of any one of claims 42 to 51, wherein the antibody comprises a V_{H} region comprising an amino acid sequence that is at least 90% identical to the amino acid sequence of SEQ ID NO: 3 and a V_{L} region comprising an amino acid sequence that is at least 90% identical to the amino acid sequence of SEQ ID NO: 4, wherein the V_{H} region comprises three CDRs comprising the amino acid sequences of SEQ ID NOs: 5-7, and the V_{L} region comprises three CDRs comprising the amino acid sequences of SEQ ID NOs: 8-10.

53. The method of any one of claims 42 to 52, wherein the antibody comprises a V_{H} region comprising the amino acid sequence of SEQ ID NO: 3 and a variable light chain V_{L} region comprising the amino acid sequence of SEQ ID NO: 4.

54. The method of any one of claims 42 to 53, wherein the antibody comprises a heavy chain comprising the amino acid sequence of SEQ ID NO: 1 and a light chain comprising the amino acid sequence of SEQ ID NO: 2.

55. The method of any one of claims 42 to 54, wherein the cancer is selected from the group consisting of: gastric cancer, melanoma (e.g., skin cutaneous melanoma), urothelial cancer, lymphoid neoplasm, diffuse large B-cell lymphoma (DLBCL), testicular germ cell tumors (TGCT), mesothelioma, kidney cancer (e.g., kidney renal clear cell carcinoma, kidney renal papillary cell carcinoma, or renal cell carcinoma (RCC)), sarcoma, lung cancer (e.g., lung adenocarcinoma, lung squamous cell carcinoma, and non-small cell lung cancer (NSCLC)) stomach adenocarcinoma, pancreatic adenocarcinoma, head and neck squamous cell carcinoma, ovarian serious cystadenocarcinoma, liver hepatocellular carcinoma, skin cutaneous melanoma, colon adenocarcinoma, breast cancer (e.g., breast invasive carcinoma or triple negative breast cancer), rectum adenocarcinoma, glioblastoma multiforme, uterine corpus endometrial carcinoma, thymoma, bladder cancer, endometrial cancer, Hodgkin's lymphoma, ovarian cancer, anal cancer, biliary cancer, colorectal cancer, and esophageal cancer.

56. The method of any one of claims 42 to 55, further comprising administration of one or more additional therapeutic agents to the subject.
